(19) United States
(12) Patent Application Publication
(10) Pub. No.: US 2025/0114144 A1
(43) Pub. Date: Apr. 10, 2025

(54) PATIENT-SPECIFIC TOTAL HIP ARTHROPLASTY DISLOCATION RISK CALCULATOR

(71) Applicant: Mayo Foundation for Medical Education and Research, Rochester, MN (US)

(72) Inventors: Bardia Khosravi, Rochester, MN (US); Pouria Rouzrokh, New Haven, CT (US); Hilal Maradit Kremers, Rochester, MN (US); Michael J. Taunton, Rochester, MN (US); Rafael J. Sierra, Rochester, MN (US); Cody C. Wyles, Rochester, MN (US)

(21) Appl. No.: 18/906,766

(22) Filed: Oct. 4, 2024

Related U.S. Application Data
(60) Provisional application No. 63/542,826, filed on Oct. 6, 2023.

(51) Int. Cl. A61B 34/10; A61F 2/46; G16H 10/60; G16H 50/30

(52) U.S. Cl. CPC A61B 34/10; A61F 2/4607; A61F 2/4609; G16H 10/60; G16H 50/30; A61F 2002/4633

(57) ABSTRACT

Disclosed herein are computer-implemented methods, and systems for performing such a methods, comprising: obtaining values for one or more non-modifiable risk factors of a patient; obtaining first candidate values for one or more modifiable risk factors of the patient; obtaining second candidate values for the one or more modifiable risk factors of the patient, wherein the second candidate values are determined to maximize the likelihood that the dislocation results from the arthroplasty procedure; determining a personalized risk interval that represents a range of modifiable risk for the patient with respect to the arthroplasty procedure, including determining (i) a lower bound for the personalized risk interval and (ii) an upper bound for the personalized risk interval; and providing an output indicative of the risk interval.

### CROSS-REFERENCE TO RELATED APPLICATIONS

This application claims the benefit of priority to U.S. Provisional Application No. 63/542,826, filed on Oct. 6, 2023. The disclosure of the prior application is considered part of the disclosure of the present document and is incorporated by reference in its entirety.

### BACKGROUND

### 1. Technical Field

This specification generally describes techniques for generating personalized dislocation risk assessments associated with total hip arthroplasty, including techniques for determining dislocation risk levels corresponding to combinations of non-operative and operative risk factors.

### 2. Background Discussion

Implant dislocation is the most common complication associated with total hip arthroplasty (THA). Oftentimes, patients become “chronic dislocators,” requiring multiple reductions by a health care professional and possibly revision surgery. In fact, implant dislocation is the leading cause of revision THA in the United States.

### SUMMARY

This specification describes systems, methods, devices, and techniques for determining personalized risk assessments for dislocations resulting from arthroplasty procedures such as THA.

In general, an aspect disclosed herein is a computer-implemented method, including obtaining values for one or more non-modifiable risk factors of a patient, each non-modifiable risk factor defining an immutable patient characteristic that is determined to impact a likelihood that a dislocation results from an arthroplasty procedure that is planned for the patient; obtaining first candidate values for one or more modifiable risk factors of the patient, each modifiable risk factor defining a mutable characteristic of the patient or the arthroplasty procedure that is determined to impact the likelihood that the dislocation results from the arthroplasty procedure that is planned for the patient, where the first candidate values are determined to minimize the likelihood that the dislocation results from the arthroplasty procedure; obtaining second candidate values for the one or more modifiable risk factors of the patient, where the second candidate values are determined to maximize the likelihood that the dislocation results from the arthroplasty procedure; determining a personalized risk interval that represents a range of modifiable risk for the patient with respect to the arthroplasty procedure, including determining (i) a lower bound for the personalized risk interval based on the values for the one or more non-modifiable risk factors of the patient and the first candidate values for the one or more modifiable risk factors and (ii) an upper bound for the personalized risk interval based on the values for the one or more non-modifiable risk factors of the patient and the second candidate values for the one or more modifiable risk factors; and providing an output indicative of the risk interval.

Examples may include one or more of the following features. Determining the lower bound for the personalized risk interval may include processing, with a machine-learning model, the values for the one or more non-modifiable risk factors of the patient and the first candidate values for the one or more modifiable risk factors; and determining the upper bound for the personalized risk interval may include processing, with the machine-learning model, the values for the one or more non-modifiable risk factors of the patient and the second candidate values for the one or more modifiable risk factors. The machine-learning model may include at least one of a regression model, an artificial neural network, a transformer model, or an XGBoost model. The arthroplasty procedure can be a total hip arthroplasty procedure and the one or more non-modifiable risk factors may include at least one of a THA indication of the patient, a sex of the patient, a body mass index of the patient, an age of the patient, an indication of a neurologic disease of the patient, a diagnosis of spine disease of the patient, a history for spine surgery of the patient, a surgery indication of the patient, or a comorbidity of the patient. The sex of the patient can be associated with values that include male and female; the age of the patient can be associated with values that include a number of years or decades since the patient's birth; the THA indication of the patient can be associated with values that include primary THA or revision THA; the body mass index of the patient can be associated with values that include less than or equal to 18, greater than 18 and less than or equal to 25, greater than 25 and less than or equal to 30, greater than 30 and less than or equal to 35, greater than 35 and less than or equal to 40, or greater than 40; the indication of a neurologic disease can be associated with values that include Parkinson disease, dementia, alcoholism, or fibromyalgia; and the surgery indication of the patient can be associated with values that include osteoarthritis, osteonecrosis, inflammation, posttraumatic, or nonunion. The arthroplasty procedure can be a total hip arthroplasty procedure and the one or more modifiable risk factors may include at least one of a femoral head diameter, a type of acetabular liner, a type of revised component, or a surgical approach. The femoral head diameter can be associated with values of cemented and non-cemented; the surgical approach can be associated with values of posterior, lateral, direct anterior, or trochanteric osteotomy; the type of acetabular liner can be associated with values of standard, elevated, constrained, or dual-mobility; and the type of revised component can be associated with values of acetabular component, femoral component, or both components. The computer-implemented method may include for each of the one or more modifiable risk factors: identifying a first value of the modifiable risk factor associated with a lowest risk of the dislocation from the arthroplasty procedure among all possible values for the modifiable risk factor; and selecting the first value for inclusion in the first candidate values. The computer-implemented method may include for each of the one or more modifiable risk factors: identifying a second value of the modifiable risk factor associated with a highest risk of the dislocation from the arthroplasty procedure among all possible values for the modifiable risk factor; and selecting the second value for inclusion in the second candidate values. The patient can be a human. The computer-implemented method may include obtaining a set of user-specified values for the one or more modifiable risk factors of the patient; and determining a personalized, modifiable risk score for the patient based on the values for the one or more non-modifiable risk factors of the patient and the set of user-specified values for the one or more modifiable risk factors. Providing the output indicative of the risk interval may include displaying an indication of the risk interval, storing the indication of the risk interval, or transmitting the indication of the risk interval to a remote computing system. Providing the output indicative of the risk interval may include generating computer code may include instructions that, when executed, cause an indication of the risk interval to be presented in an interactive user interface on a screen of an electronic device. The arthroplasty procedure can be a total hip arthroplasty procedure and the one or more non-modifiable risk factors may include image features extracted from one or more pre-operative images of a femoral or pelvic region of the patient. The computer-implemented method may include extracting the image features using a machine-learning model trained to predict whether or a likelihood that a patient exhibits dislocation following the total hip arthroplasty procedure based on one or more pre-operative images of the femoral or pelvic region.

In general, an aspect disclosed herein is one or more non-transitory computer-readable media encoded with instructions that, when executed by one or more processors, cause the one or more processors to perform any of the aspects disclosed herein.

In general, an aspect disclosed herein is a system, including: one or more processors; and one or more computer-readable media encoded with instructions that, when executed by the one or more processors, cause the one or more processors to perform any of the aspects disclosed herein.

In general, an aspect disclosed herein is a computer-implemented method, including obtaining user-indicated values for one or more non-modifiable risk factors of a patient, each non-modifiable risk factor defining an immutable patient characteristic that is determined to impact a likelihood that a dislocation results from an arthroplasty procedure that is planned for the patient; obtaining user-indicated values for one or more modifiable risk factors of the patient, each modifiable risk factor defining a mutable characteristic of the patient or the arthroplasty procedure that is determined to impact the likelihood that the dislocation results from the arthroplasty procedure that is planned for the patient; determining a personalized, modifiable risk score for the patient with respect to the dislocation and the arthroplasty procedure based on the user-indicated values for the one or more non-modifiable risk factors of the patient and the user-indicated values for the one or more modifiable risk factors; and providing an output indicative of the personalized, modifiable risk score for the patient.

Examples may include one or more of the following features. Determining the personalized, modifiable risk score may include processing, with a machine-learning model, the user-indicated values for the one or more non-modifiable risk factors of the patient and the user-indicated values for the one or more modifiable risk factors. The arthroplasty procedure can be a total hip arthroplasty procedure and the one or more non-modifiable risk factors may include at least one of a THA indication of the patient, a sex of the patient, a body mass index of the patient, an age of the patient, an indication of a neurologic disease of the patient, a diagnosis of spine disease of the patient, a history for spine surgery of the patient, a surgery indication of the patient, or a comorbidity of the patient. The arthroplasty procedure can be a total hip arthroplasty procedure and the one or more non-modifiable risk factors may include image features extracted from one or more pre-operative images of a femoral or pelvic region of the patient. The computer-implemented method may include extracting the image features using a machine-learning model trained to predict whether or a likelihood that a patient exhibits dislocation following the total hip arthroplasty procedure based on one or more pre-operative images of the femoral or pelvic region.

In general, an aspect disclosed herein is one or more non-transitory computer-readable media encoded with instructions that, when executed by one or more processors, cause the one or more processors to perform any of the methods disclosed herein.

In general, an aspect disclosed herein is a system, including one or more processors; and one or more computer-readable media encoded with instructions that, when executed by the one or more processors, cause the one or more processors to perform any of the methods disclosed herein.

Additional features and advantages will be apparent to one of ordinary skill in view of the specification, the figures, and claims.

### BRIEF DESCRIPTION OF DRAWINGS

FIG. 1 is a schematic diagram of an example system for presenting calculated dislocation risk associated with total hip arthroplasty on a user device.

FIG. 2 is an image depicting an example user interface for the inputting information relating to non-modifiable medical information for calculating a dislocation risk nomogram.

FIG. 3A is an image depicting an example user interface for viewing the dislocation risk nomogram based on entered non-modifiable medical information.

FIG. 3B is an image depicting an example user interface for viewing another dislocation risk nomogram.

FIG. 4 is a waterfall Shapley additive explanation (SHAP) plot for the patient presented in FIG. 3B with a combination of standard acetabular liner, femoral head component smaller than 32 mm, and posterior approach.

FIG. 5 is a waterfall Shapley additive explanation (SHAP) plot for the patient presented in FIG. 3B with a combination of dual-mobility acetabular liner, femoral head component larger than 36 mm, and direct anterior approach.

FIG. 6 is a flow chart diagram depicting a process an example computer-implemented process for determining a personalized risk interval.

FIG. 7 is a flow chart diagram depicting a process for determining a personalized, modifiable risk score for a patient with respect to a complication and an arthroplasty procedure.

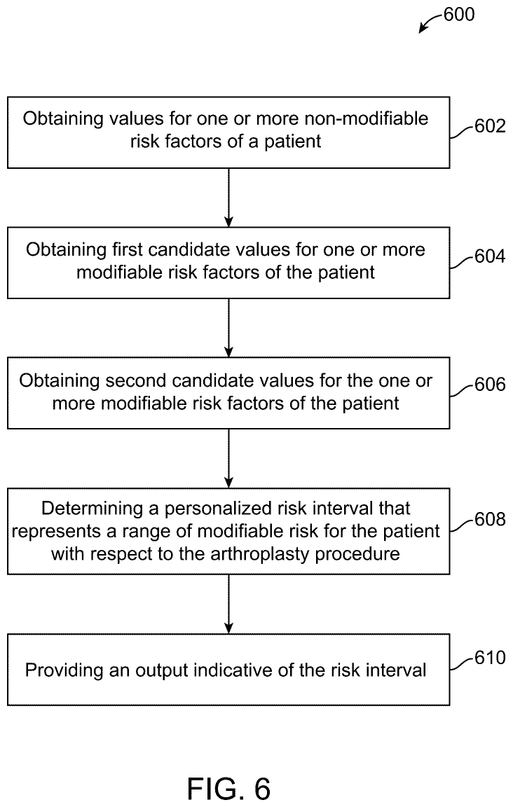

FIG. 6 is a table showing patient profiles with non-modifiable preoperative variables and modifiable intraoperative variables.

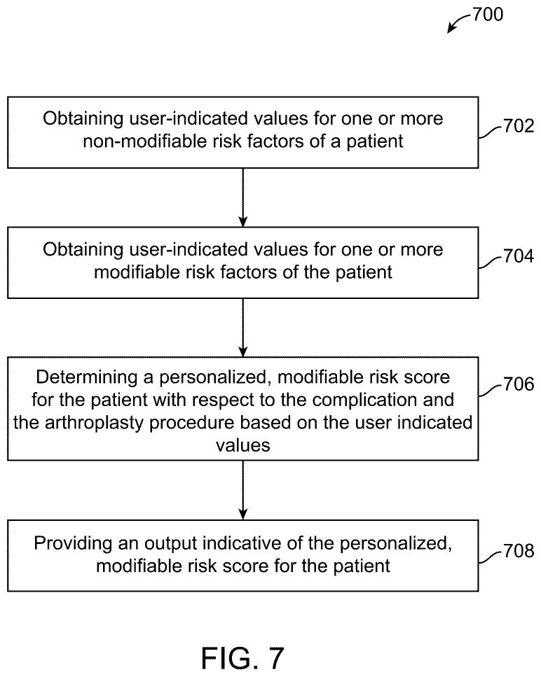

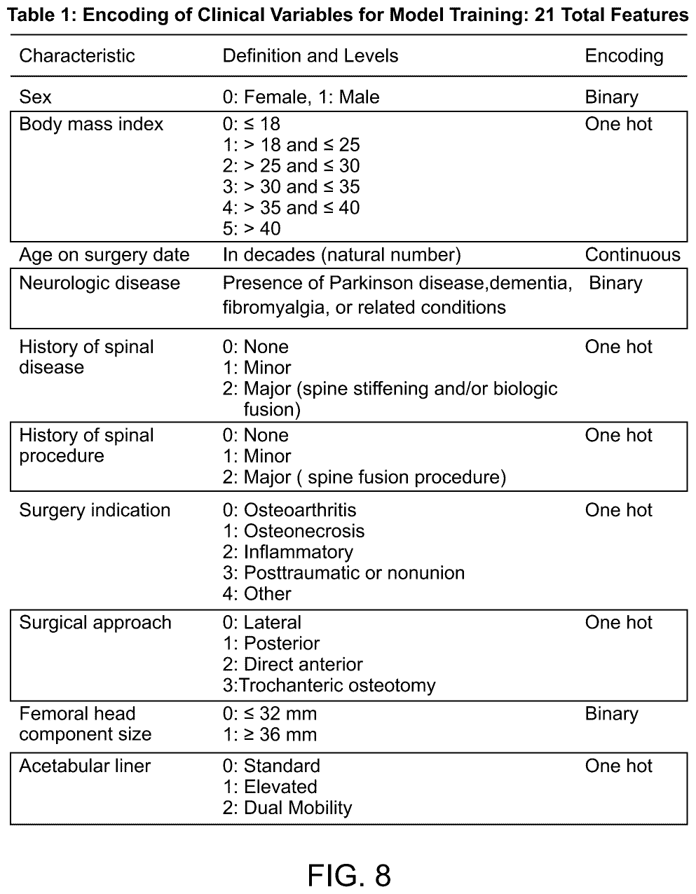

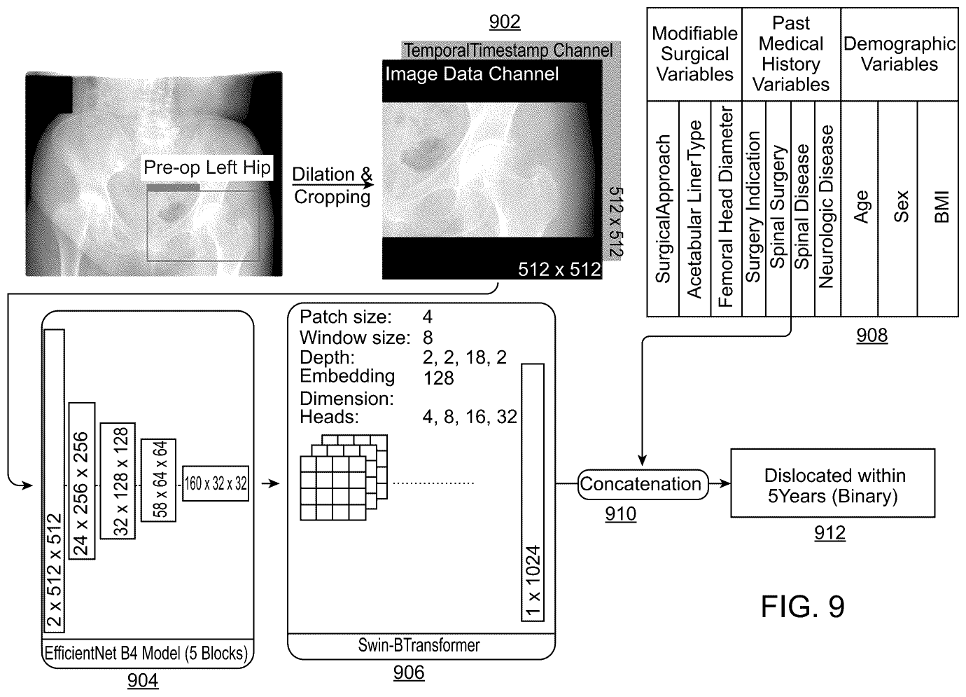

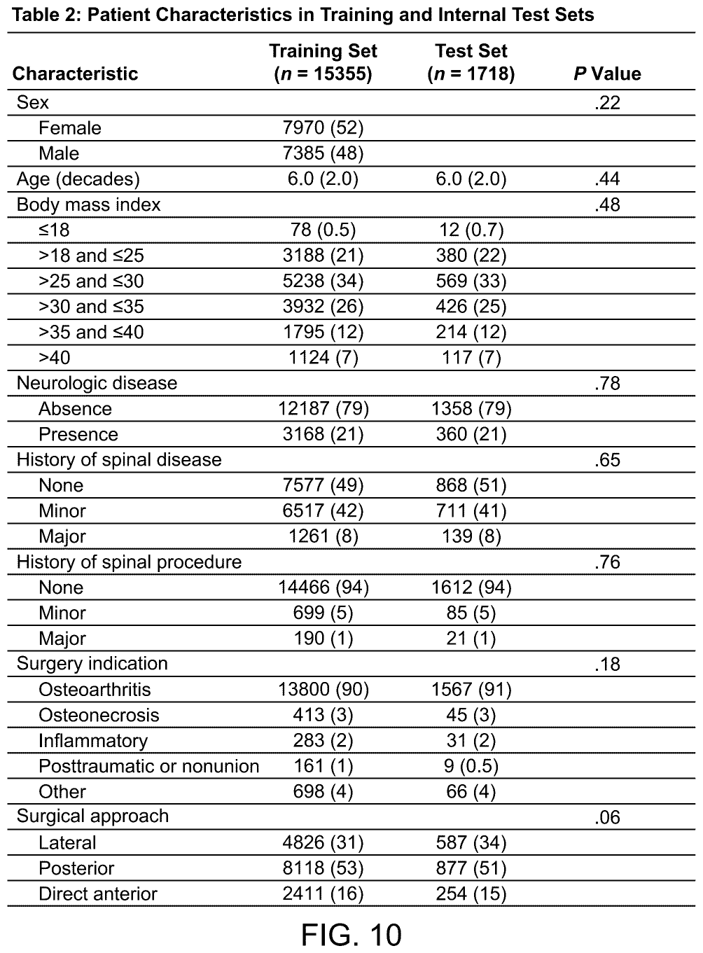

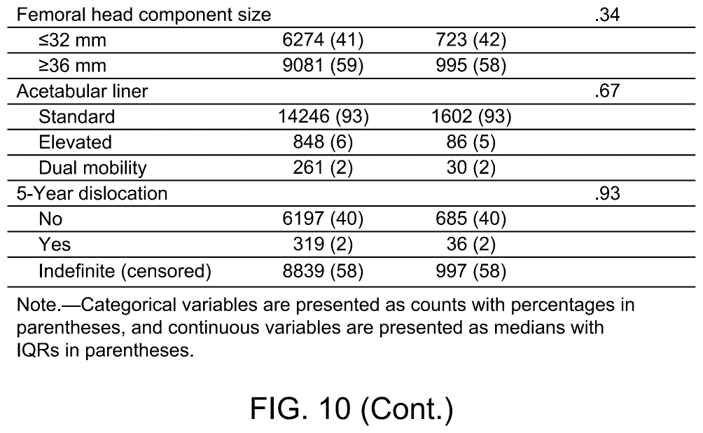

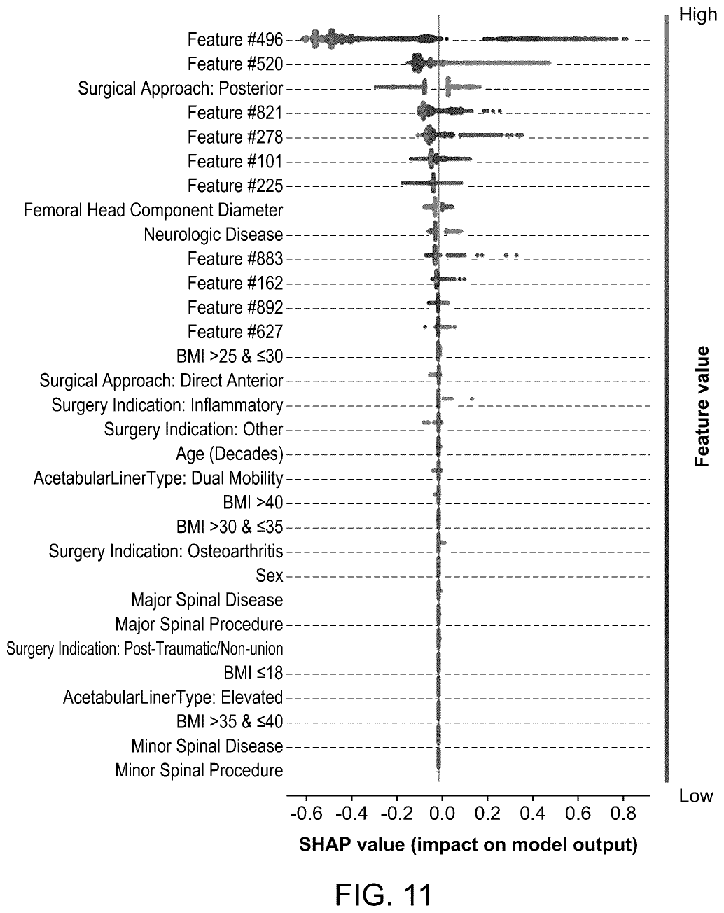

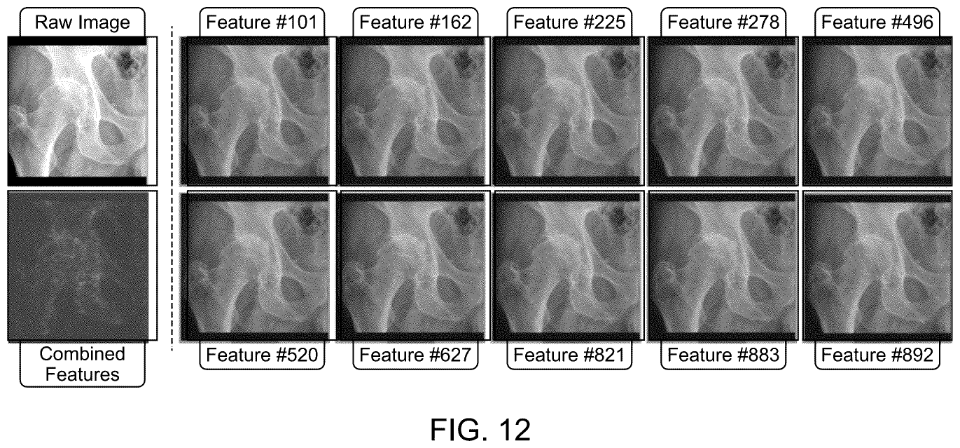

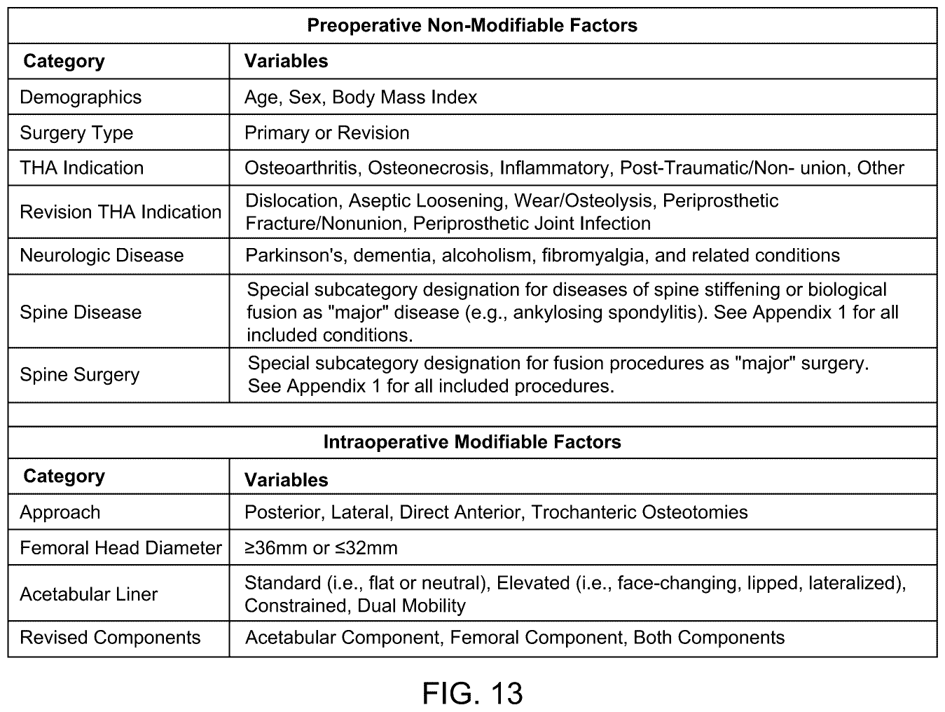

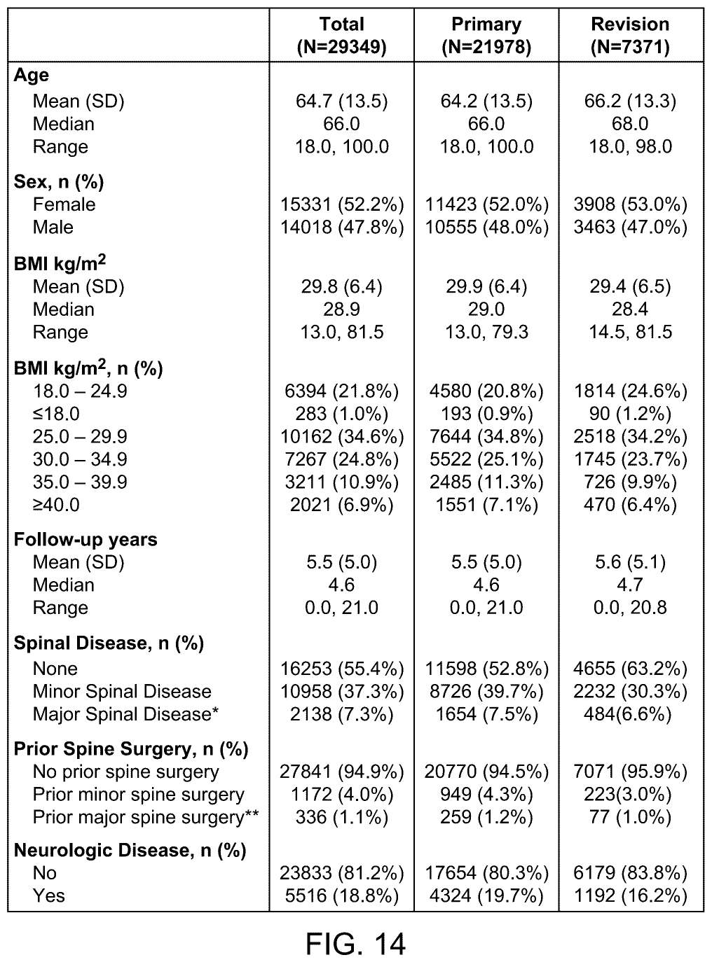

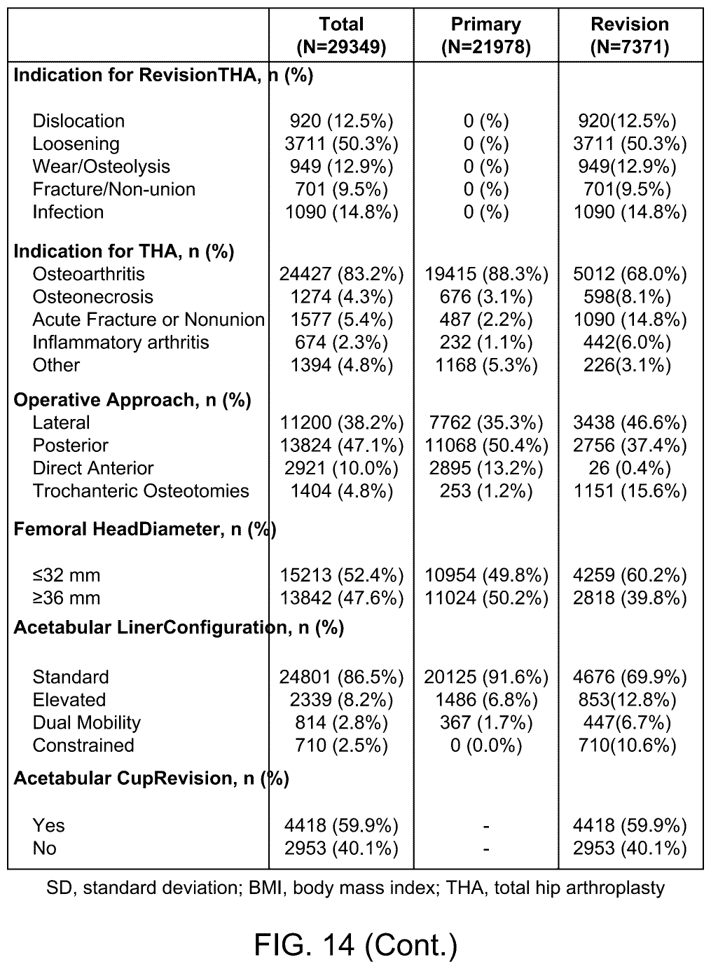

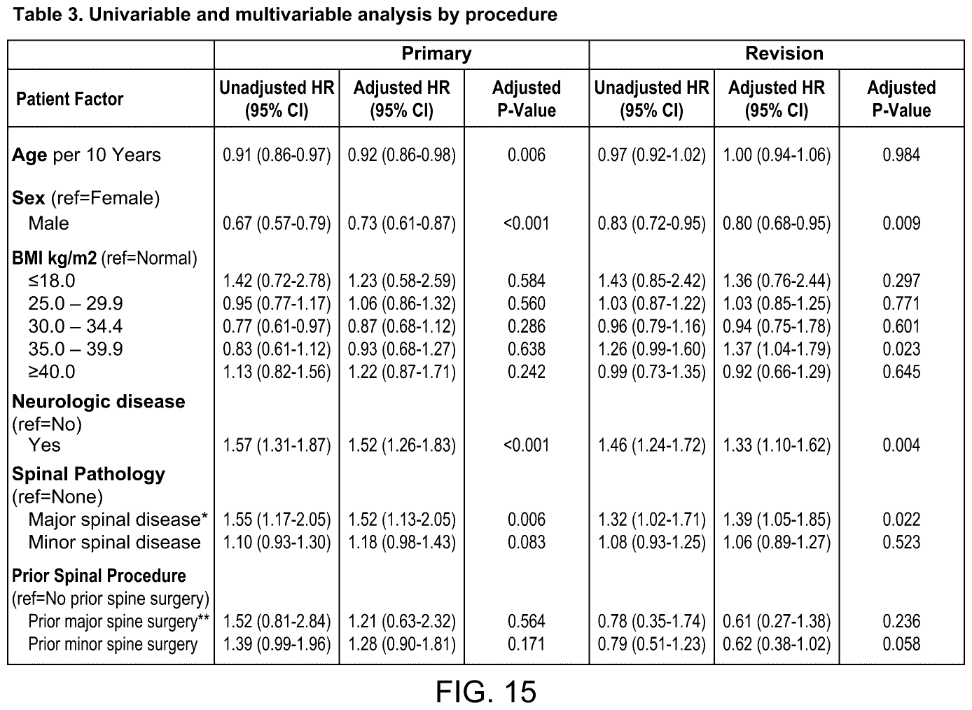

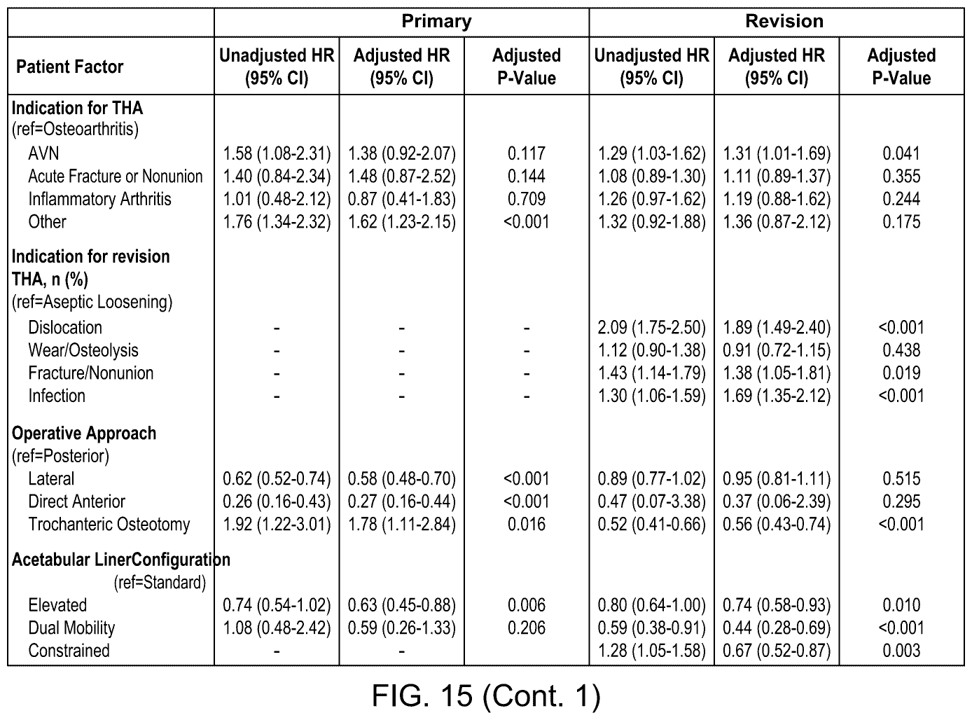

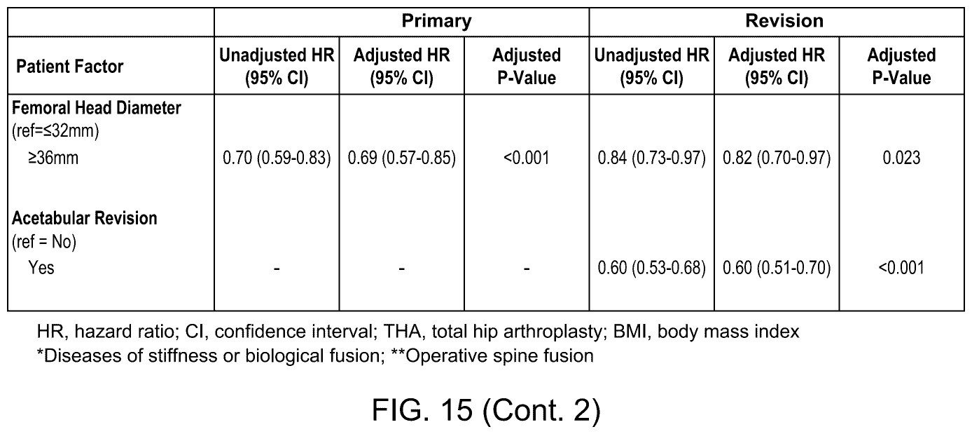

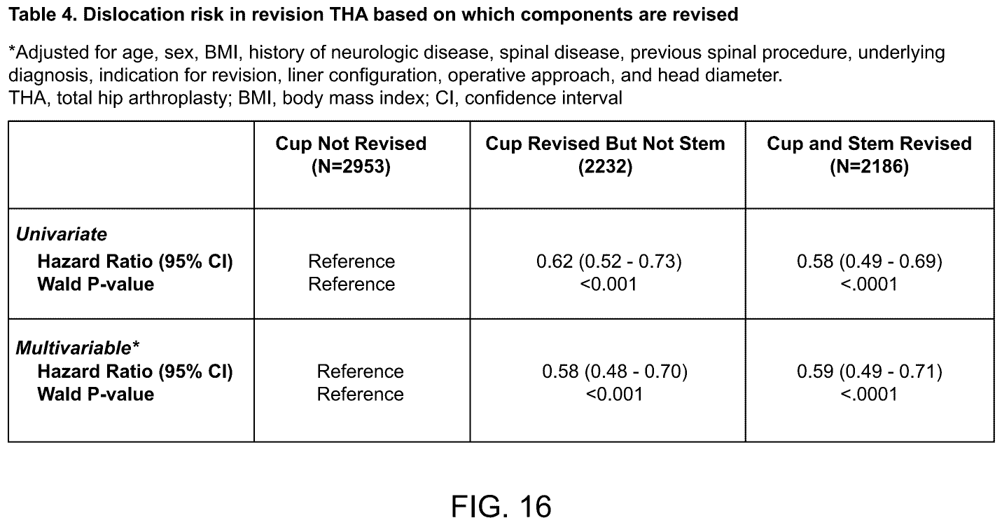

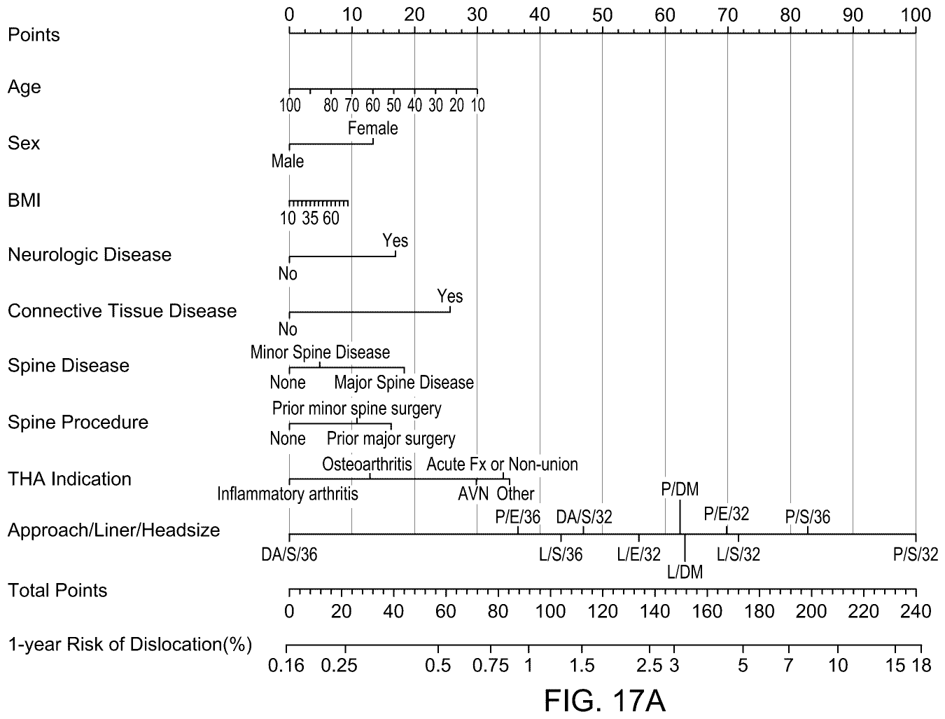

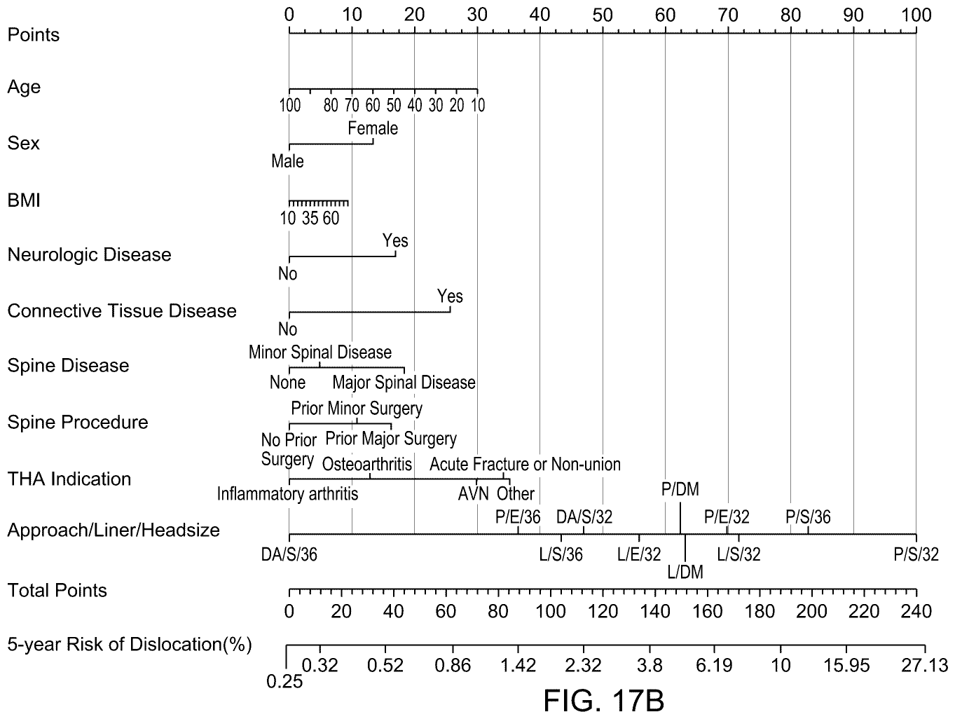

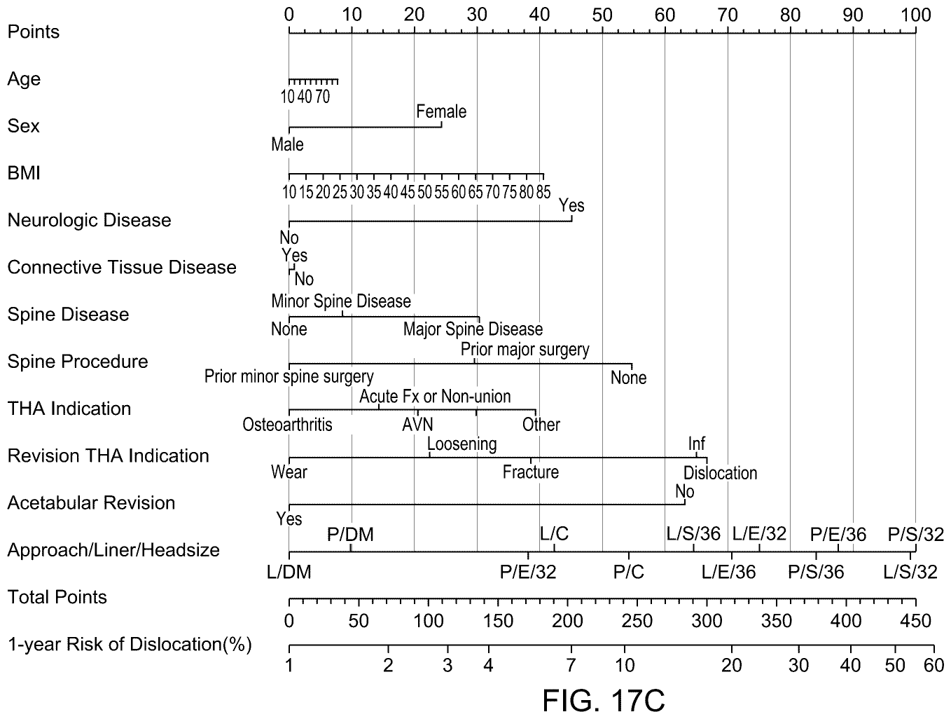

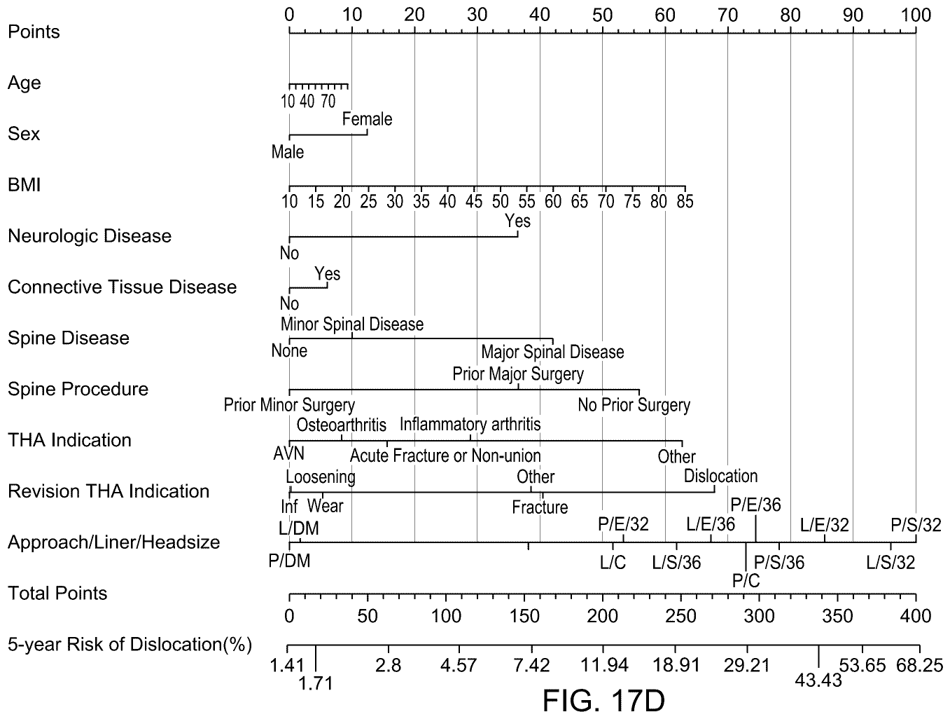

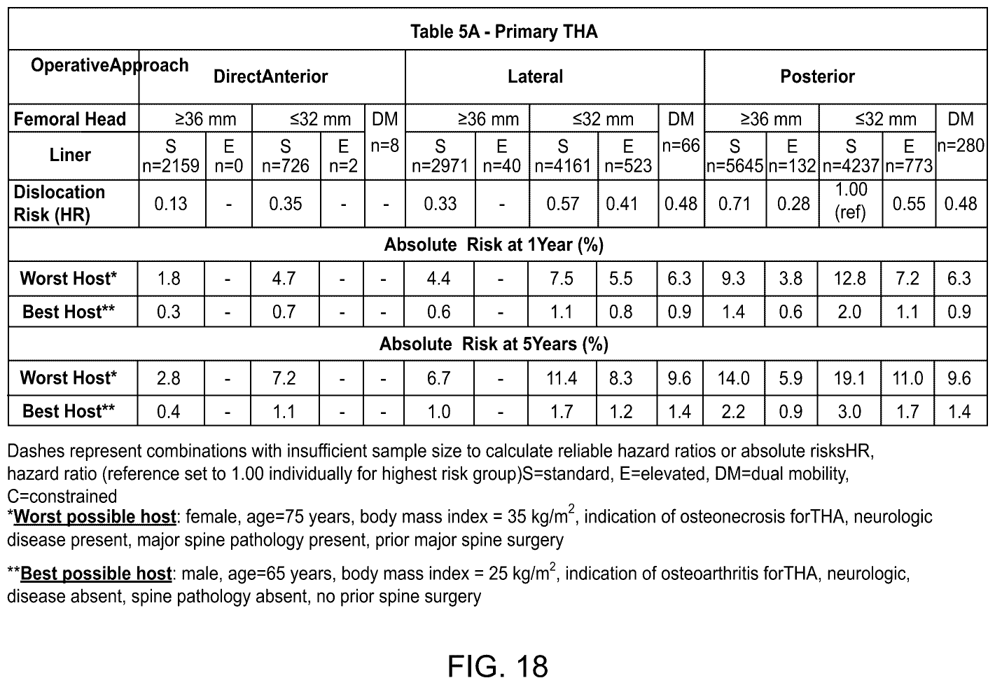

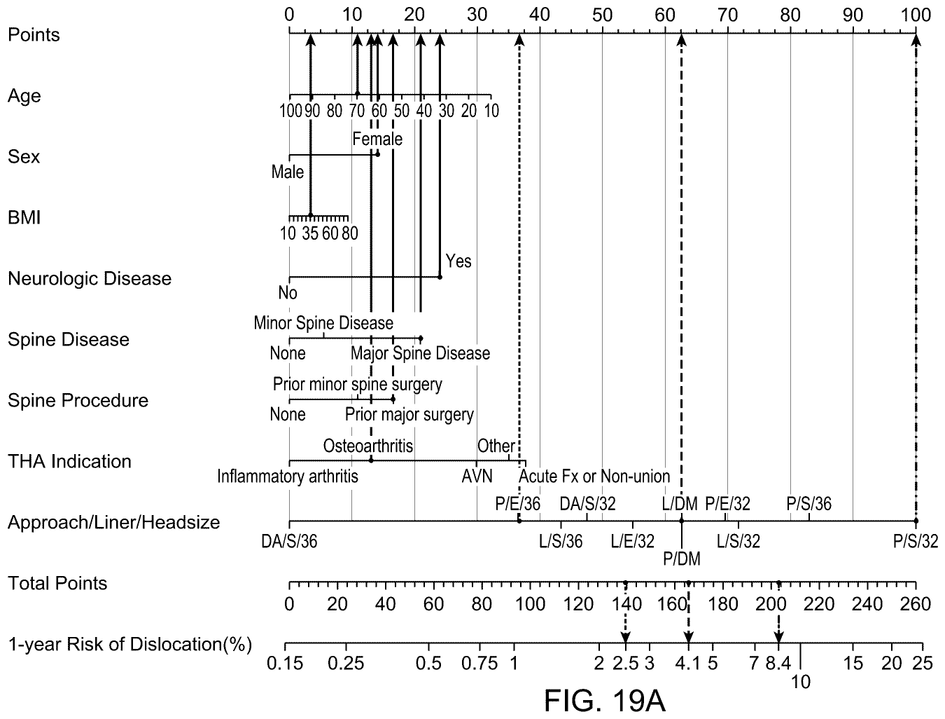

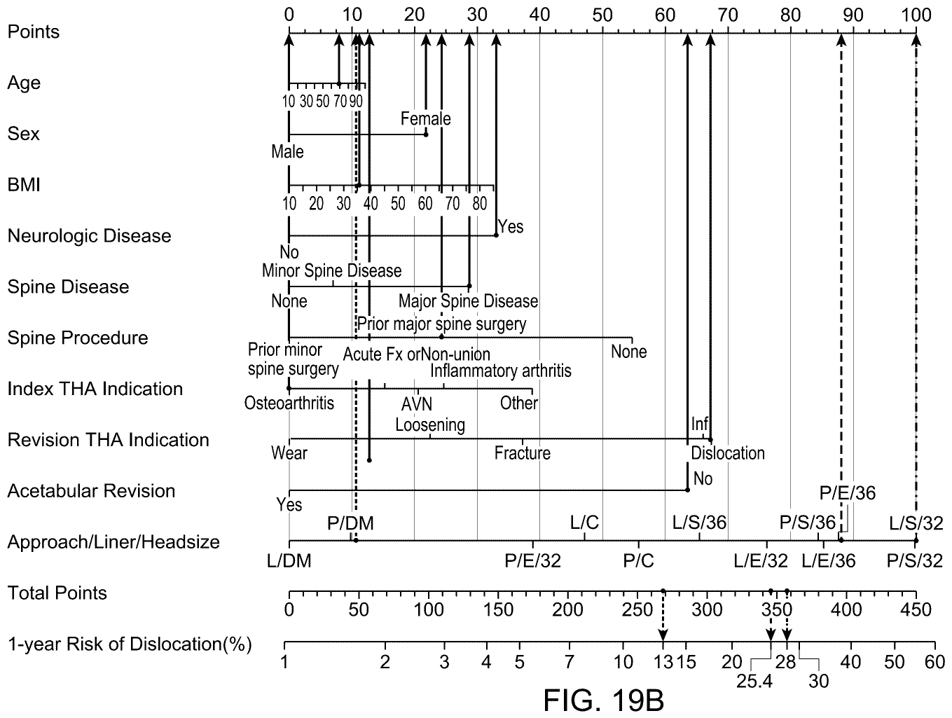

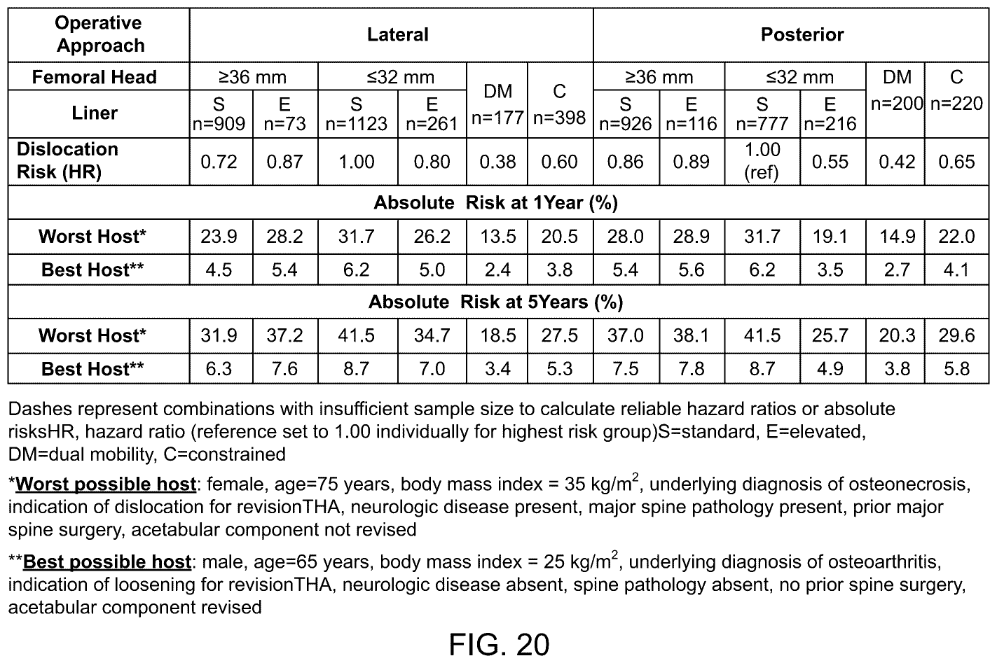

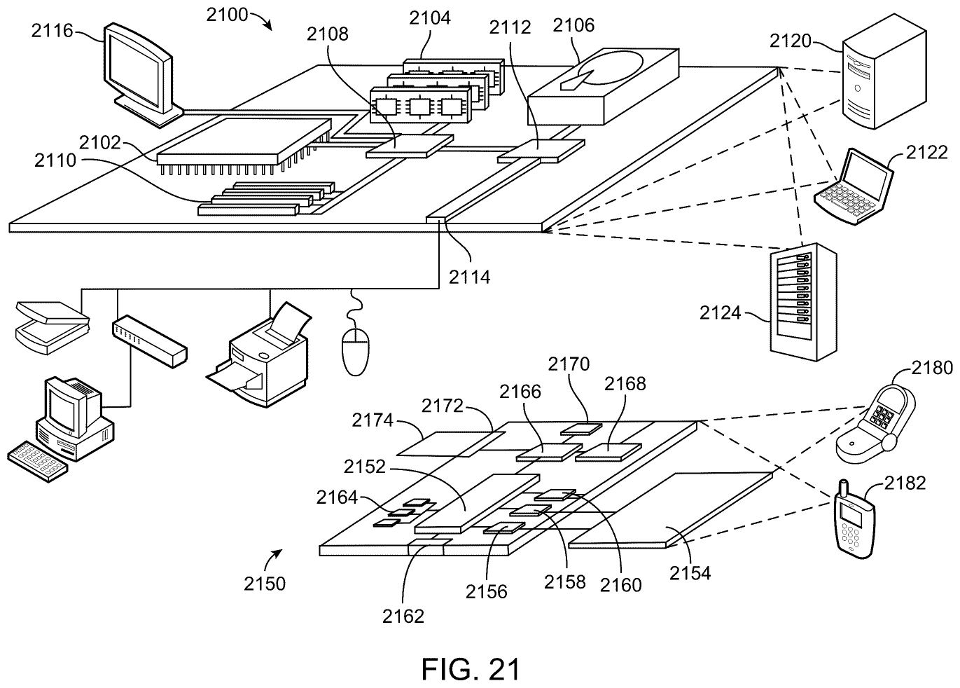

FIG. 7 is a table showing patient characteristics and primary THA performed for the patients of the study.

FIG. 8 is a table showing evaluated patient factors encoded as clinical variables for model training.

FIG. 9 is a flow chart diagram depicting an architecture for the deep learning multimodal classifier.

FIG. 10 is a table showing evaluated patient factors included in the final univariable and multivariable risk analysis models.

FIG. 11 is a SHAP plot of the trained survival model.

FIG. 12 is a series of images depicting integrated gradient maps of selected features.

FIG. 13 is a table showing determinated patient profiles with non-modifiable preoperative variables and modifiable intraoperative variables.

FIG. 14 is a table showing baseline characteristics for the overall cohort as well as the primary and revision subgroups.

FIG. 15 is a table showing results of the univariable and multivariable analyses of dislocation risk associated with patient factors.

FIG. 16 is a table showing dislocation risk after revision THA based on which component(s) were revised.

FIGS. 17A-B are individual-patient dislocation risk nomograms for primary total hip arthroplasty at 1 year and 5 years, respectively.

FIGS. 17C-D are individual-patient dislocation risk nomograms for revision total hip arthroplasty at 1 year and 5 years, respectively.

FIG. 18 is a table showing dislocation risk after primary THA based on non-modifiable patient factors and modifiable operative decisions.

FIGS. 19A-19B are nomograms for hypothetical patients and various risk estimates in the setting of a primary THA (A) and revision THA (B), respectively, including exemplary lines depicting how to use the nomogram.

FIG. 20 is a table dislocation risk after revision THA based on non-modifiable patient factors and modifiable operative decisions.

FIG. 21 is a schematic diagram of example computer systems for executing the methods and systems described herein.

### DETAILED DESCRIPTION

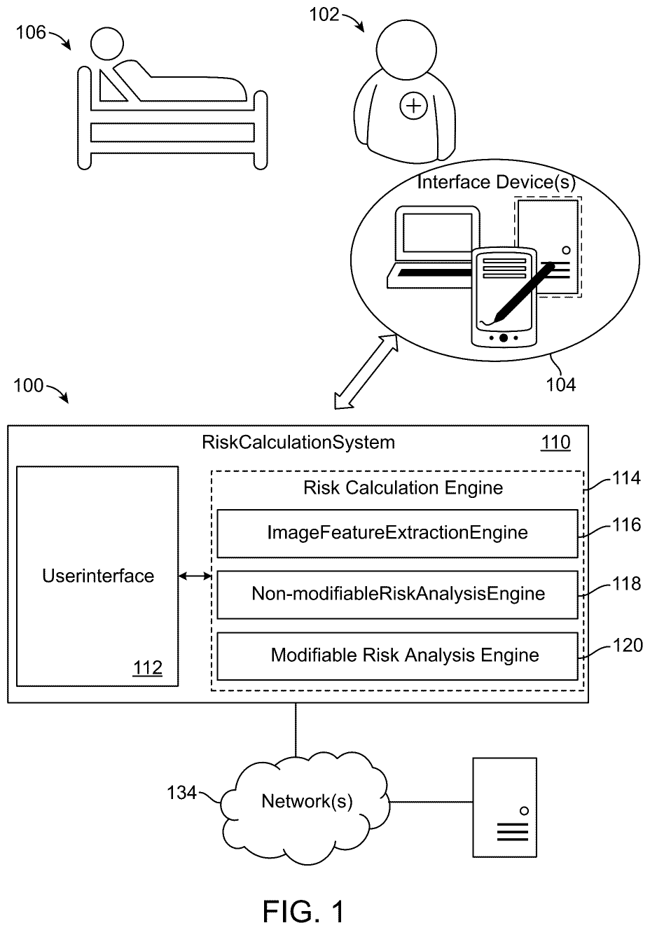

FIG. 1 is a block diagram of an example patient-specific dislocation risk prediction system 100 configured to dynamically generate dislocation risk assessments based on data indicative of both non-operative data and operative conditions of a patient with respect to an arthroplasty procedure. An operative decision is a discrete, modifiable aspect of a planned arthroplasty procedure that can be pre-operatively selected and that can affect the risk of a negative outcome resulting from the procedure, such as a dislocation following total hip arthroplasty. In this specification, the term ‘operative’ refers to a class of ‘modifiable’ risk factors and the term ‘non-operative’ refers to a class of ‘non-modifiable’ risk factors. The example risk prediction system 100 can be configured to determine the risk of dislocation resulting from an arthroplasty procedure which is planned for a patient.

System 100 depicts a user 102 interacting with an example interface device 104. The user 102 in this example is a clinician, e.g., a medical professional, a surgeon, or medical assistant, although the techniques disclosed in this specification may be extended for use with other users as well. In the example of FIG. 1, the user 102 is screening a patient 106 for an arthroplasty procedure, e.g., a THA procedure, and utilizing the system 100 to determine a personalized risk interval, e.g., a nomogram, based on one or more non-modifiable and one or more modifiable, operable risk factors which can be made by the user 102 pre-operatively.

The user 102 interacts with the interface device 104 to input user-specified, non-modifiable risk factors into the system 100. The interface device 104 stores in non-transitory media the risk calculation system 110. The risk calculation system 110 includes a user interface 112 with which the user 102 interacts and inputs the non-modifiable risk factors into the model 110. The user interface 112 includes control elements for receiving the input from the user 102 such as radio buttons, text boxes, and/or other input fields into which the user 102 inputs the medical data for the patient 106.

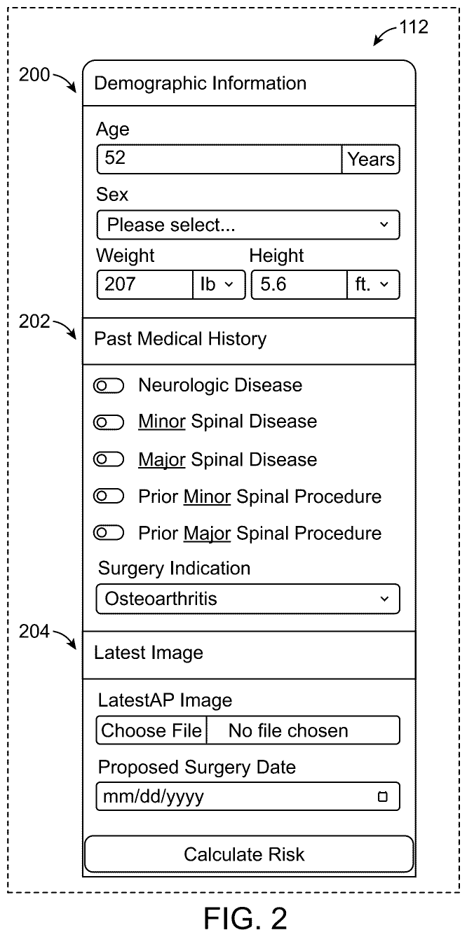

Referring to FIG. 2, exemplary user interface windows are shown which can be presented to the user 102 on the user interface 112. The exemplary user interface windows of FIG. 2 include a demographic information window 200, a past medical history window 202, and an image upload window 204. The demographic information window 200 includes input fields for some non-modifiable risk factors which can be user-specified values, or values received from a patient data management platform. The non-modifiable risk factors displayed in the demographic information window 200 are age, sex, weight, and height.

The past medical history window 202 includes binary input fields (e.g., binary sliders, radio buttons) for non-modifiable risk factors which have a binary value indicating the presence or absence of the associated non-modifiable risk factors. As with other non-modifiable risk factors, the binary risk factors can be user-specified values or received from a patient data management platform. The binary non-modifiable risk factors displayed in the demographic information window 200 can include binary indicators as to whether the patient has or has had neurologic disease, minor spinal disease, major spinal disease, prior minor spinal procedure, prior major spinal procedure, and/or indications of osteoarthritis.

The values displayed in the demographic information window 200 and/or the medical history window 202 can be the values input into the user interface 112 in the demographic information window 200 or the medical history window 202, respectively.

The image upload window 204 includes a file selection field in which a user may select one or more image files containing image data for processing by the risk prediction system 100. The image file can be any medically-relevant image file, such as JPEG, PNG, or other medically-related image file which the image feature extraction engine 116 can process the image data and extract features from. For example, in a THA procedure, the user 102 may upload image files containing image data representing medical scans of a hip area of a patient detailing portions of the femoral head, femoral stem, or bones of the pelvic girdle.

Referring again to FIG. 1, the user interface 112 receives the input indicative of non-operable medical data of the patient 106 and transmits the medical data to a risk calculation engine 114 which communicates with an image feature extraction engine 116, a non-modifiable risk calculation engine 118, and a modifiable risk calculation engine 120.

In another example, the system 100 receives non-modifiable patient data from a database, look up table, or other data storage system connected to a network 134 in communication with the system 100, such as a patient data management system which stores individualized, non-modifiable risk factors specific to the patient 106. The system 100 can receive the non-modifiable patient data from the network 134 alone or in combination with user-specified, non-modifiable risk factors input by the user 102.

The user interface 112 is communicatively connected to a risk calculation engine 114 which receives the modifiable and non-modifiable risk factors from the user interface 112. The risk calculation engine 114 includes an image feature extraction engine 116, a non-modifiable risk calculation engine 118, and a modifiable risk calculation engine 120. In some cases, the image feature extraction engine 116 is implemented in the non-modifiable risk calculation engine 118 since the image features can be considered non-modifiable risk factors. Each of the engines 116, 118, 120 can be implementations of suitable models trained to generate output based on the received input. In some examples, the models are machine-learning models.

The image feature extraction engine 116 is configured to receive pre-operative images, e.g., image data, from the user interface 112 and generate image features indicative of one or more risk factors of the procedure. The image feature extraction engine 116 receives image data and generates a set of features based on the received images which can be correlated with one or more surgical outcomes related to the modifiable options or non-modifiable risk factors. Examples of the image feature extraction engine 116 are survival machine-learning models, or multimodal survival machine-learning models which can process more than one type of input data.

One example of the image feature extraction engine 116 is XGBoost, an open-source implementation of the supervised learning, gradient-boosted trees algorithm which attempts to accurately predict a target variable by combining the estimates of a set of simpler, weaker models. The image feature extraction engine 116 can also be implemented with convolutional neural networks, transformers, or other machine-learning models. The image feature extraction engine 116 extracts the most informative imaging features from the image data and concatenates the image data with one or more clinical features from the received modifiable or non-modifiable patient data. The image feature extraction engine 116 is pretrained using a collection of these features to output predicted individualized risk of dislocation based on the features extracted from the clinical images.

The non-modifiable risk analysis engine 118 and the modifiable risk calculation engine 120 can be implementations of a multivariable regression model, e.g., a Cox proportional hazard models, configured to associate time to outcome events against a set of explanatory variables, e.g., the non-modifiable patient data. The non-modifiable risk calculation engine 118 is trained on patient data which included values for one or more of the non-modifiable risk factors. In some examples, the non-modifiable risk factors include demographics, THA indication (e.g., primary THA, and/or revision THA), sex (e.g., but not limited to, male or female), body mass index (e.g., ≤18, >18 and ≤25, >25 and ≤30, >30 and ≤35, >35 and ≤40, or >40), age (e.g., a number of years or decades since the patient's birth), neurologic disease (e.g., Parkinson disease, dementia, alcoholism, fibromyalgia, or related conditions), diagnosis of spine disease (e.g., subcategory designations of diseases causing spine stiffening or biological fusion as “major” (e.g., ankylosing spondylitis), “minor” disease, or no disease), and history for spine surgery (e.g., subcategory designations of fusion procedures as “major” surgery, “minor” surgery, or no surgery), surgery indication (e.g., surgical indications for osteoarthritis, osteonecrosis, inflammation, posttraumatic, or nonunion), or comorbidities.

The modifiable risk calculation engine 120 is trained on interoperative surgical decisions which include values for one or more of the modifiable risk factors, such as surgical approach (e.g., posterior, lateral, direct anterior, or trochanteric osteotomy), femoral head diameter (e.g., greater than or equal to 36 mm, or less than or equal to 32 mm), type of acetabular liner (e.g., standard (i.e., flat or neutral), elevated (i.e., face-changing, lipped, or lateralized), constrained, or dual-mobility), or type of revised component(s) (e.g., acetabular component, femoral component, or both components).

As an example, a hybrid network of EfficientNet-B4 and Swin-B transformer can be configured to classify patients based on n-year dislocation outcomes (e.g., 1-, 2-, 5-, or 10-year outcomes) from preoperative medical images, e.g., images of the anteroposterior (AP) pelvis radiographs, and clinical (demographics, comorbidities, and surgical) characteristics. The most informative imaging features, extracted by the mentioned model, can then be selected and concatenated with clinical features. A collection of these features can be used to train a multimodal survival XGBoost model to predict the individualized risk of dislocation or other complication of interest.

The engine 120 receive the modifiable risk factor values from the user interface 112 and generates first candidate values which represents the minimum likelihood, and second candidate values which represents the maximum likelihood, that a complication such as a dislocation results from the arthroplasty procedure.

The model 110 determines a personalized risk interval for the patient based on their values for the one or more non-modifiable risk factors with the first and second candidate values from the modifiable risk factor values. The interval is defined by an upper and a lower bound for the personalized risk interval using best and worst possible patient scenarios. The upper and lower bounds are based on the maximum and minimum complication likelihoods from the engines 118 and 120.

Based on the output from the engines 116, 118, 120, the model 110 generates an output indicative of the risk interval. The model 110 provides the output to the user interface 112 such that the risk calculation engine 114 presents the output for display to the user 102. In some examples, the model 110 provides the output to the interface device 104. Additionally or alternatively, the model 110 generates computer code including instructions that, when executed, cause an indication of the risk interval to be presented on the interface device 104.

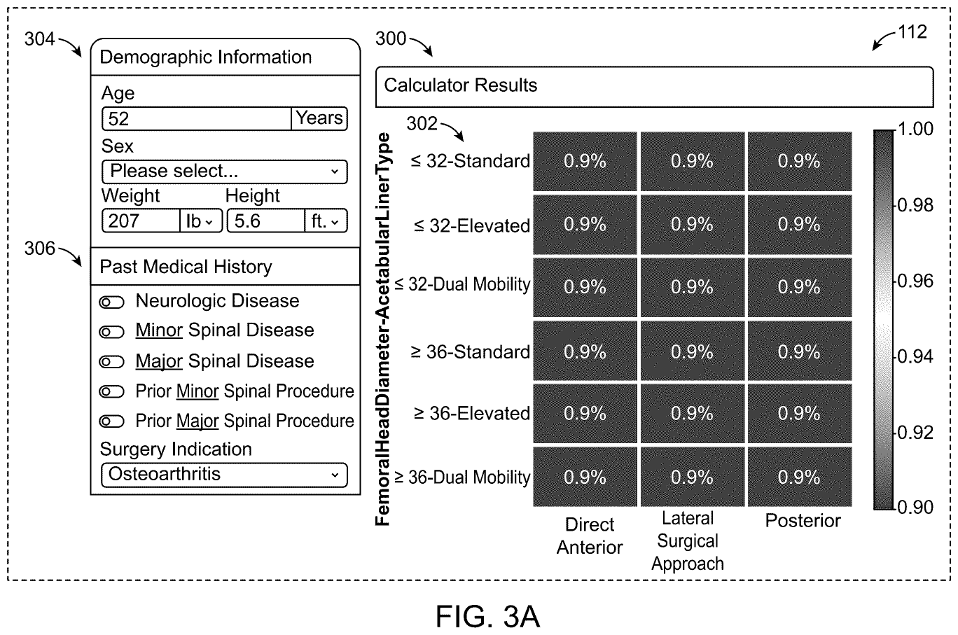

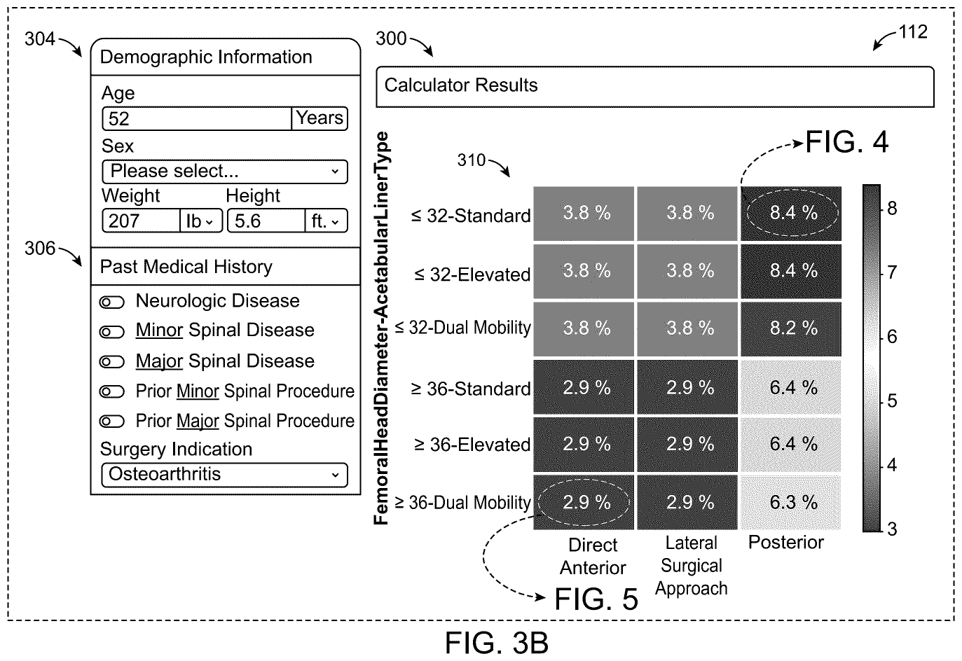

In one example, the output is a table of risk values comparing one or more of the modifiable risk factors against one or more of the remaining modifiable risk factors which can be presented on the user interface 112 of the interface device 104 to the user 102. Referring to FIG. 3A, the output of the model 110 is indicated as displayed in the user interface 112 in a results window 300 titled ‘Calculator Results’ and presents a table 302 comparing values of three operable risk factors including femoral head diameter (e.g., ≤32 mm, or ≥36 mm) and acetabular liner type (e.g., standard, elevated, or dual mobility) against surgical approach (e.g., direct anterior, lateral, or posterior). By presenting combinations of two risk factors along a first table axis against a single risk factor, the table presents an indication of which combination of three operable risk factors may lead to the least risk of negative outcomes, such as dislocation. A heat-map legend is shown adjacent the table 302, indicating lowest risk values (e.g., 0.90) at the bottom and highest risk values (e.g., 1) at the top. In this example, the table 302 demonstrates essentially no impact of surgical decisions, e.g., the combination of all risk factors is shown as 0.9%.

A demographic information window 304 is shown adjacent the results window 300 which includes some values of the user-provided demographic information such as age, sex, weight, and height. Other values of the non-modifiable risk factors may also be displayed. A medical history window 306 is shown adjacent the results window 300 and demographic information window 304 which displays user-selected non-modifiable risk factors which are represented by binary values, e.g., presence or absence of the non-modifiable risk factor. In the example medical history window 306, binary values for neurologic disease, minor spinal disease, major spinal disease, prior minor spinal procedure, prior major spinal procedure, and indications of osteoarthritis are shown.

Referring to FIG. 3B, the output of the model 110 for a second example patient is presented in table 310 comparing values of the same three risk factors as FIG. 3A, (e.g., femoral head diameter and acetabular liner type, against surgical approach method. A heat-map legend is shown adjacent the table 310, indicating lowest risk values (e.g., ≥3) at the bottom and highest risk values (e.g., ≤8.4%) at the top. In this example, the table 310 demonstrates the combination of a ≤32 femoral head diameter, regardless of acetabular liner type, with a posterior surgical approach method has the highest risk factor based on the non-modifiable risk factors of 8.4% chance of a dislocation within the selected time window. The table 310 indicates the combination of a ≥36 femoral head diameter, regardless of acetabular liner type, and either a direct anterior or a lateral surgical approach has the lowest risk factor of 2.9% chance of a dislocation within the selected time window.

In some examples, the model 110 generates an output indicative of the relative contribution of various factors to the model 110 prediction. The model 110 provides the factor contribution output to the user interface 112 for display to the user 102. In some examples, the model 110 provides the output to the interface device 104. Additionally or alternatively, the model 110 generates computer code including instructions that, when executed, cause an indication of the relative contribution of various factors to be presented on the interface device 104.

In one example, the output is a patient-specific plot comparing one or more of the modifiable or non-modifiable risk factors against the model weighting value for the risk factors on the left axis which can be presented on the user interface 112 of the interface device 104 to the user 102. One example of the output is a Shapley additive explanation (SHAP) summary plot.

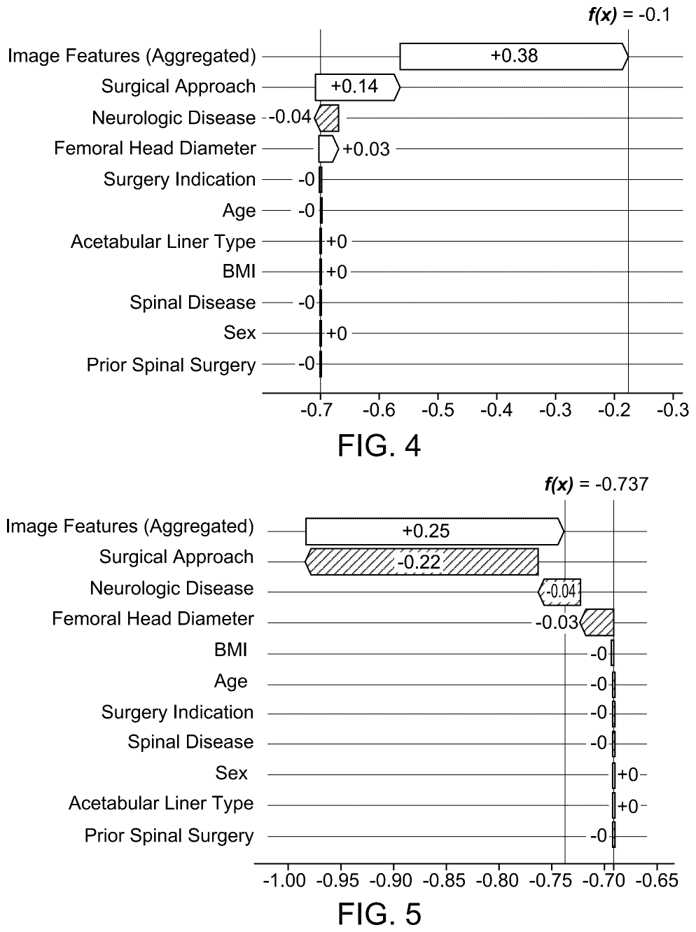

FIG. 4 is a SHAP plot for the combination of standard acetabular liner, femoral head component smaller than 32 mm, and posterior approach, e.g., the upper right matrix element in FIG. 3B. The shading pattern of the bars in the chart is determined by the feature value for the feature given on the left axis. For example, the aggregated image features determined by image feature extraction engine 116 (first pattern) is associated with higher SHAP values or, e.g., higher risk of dislocation. In another example, neurological disease indicators (second pattern) are associated with lower SHAP values, e.g., lower risk of dislocation. FIG. 4 shows the two highest relative contributions to the model 110 output include aggregated image features (+0.38) and surgical approach (+0.14), the lowest relative contribution includes neurological disease (−0.04), and negligible contributions to the model 110 output include surgery indications, age, acetabular liner type, BMI, indications of spinal disease, sex, and prior spinal surgery indications.

FIG. 5 is a SHAP plot for the combination of dual-mobility acetabular liner, femoral head component greater than 36 mm, and direct anterior approach, e.g., the lower left matrix element in FIG. 3B. The highest relative contribution to the model 110 output includes aggregated image features (+0.25), the lowest relative contribution includes surgical approach (−0.22), and negligible contributions to the model 110 output include surgery indications, age, acetabular liner type, BMI, indications of spinal disease, sex, and prior spinal surgery indications.

FIG. 6 is a flowchart of an example computer-implemented process 600 for determining a personalized risk interval that represents a range of modifiable risk for the patient with respect to the arthroplasty procedure and providing an output indicative of the risk interval.

The process 600 may be used, for example, by a medical user, e.g., user 102, for determining total risk from complications for patients, e.g., patient 106, undergoing an arthroplasty procedure, e.g., a total hip or other arthroplasty procedure. By inputting values for one or more non-modifiable or modifiable risk factors of the patient into the computer-implemented process 600, the process 600 determines a personalized risk interval that represents a range of modifiable risk for the patient with respect to the arthroplasty procedure.

A clinician or other user obtains values for one or more non-modifiable risk factors of a patient (602). In general, the patient can be a human, though in other examples, the patient is an animal. In some implementations, each non-modifiable risk factor defines an immutable patient characteristic that is determined to impact a likelihood that a complication results from an arthroplasty procedure that is planned for the patient. Examples of the non-modifiable risk factors include any described herein. Some examples of the arthroplasty procedure include a total hip arthroplasty procedure, while an example of the complication can be a dislocation.

In an example in which the arthroplasty procedure is a total hip arthroplasty procedure, and the complication is dislocation, the non-modifiable risk factor includes image features extracted from one or more pre-operative images of a femoral or pelvic region of the patient. The image features can be extracted from the pre-operative image using a machine-learning model trained to predict whether, or a likelihood that, a patient exhibits dislocation following the total hip arthroplasty procedure based on one or more pre-operative images of the femoral or pelvic region.

The medical user obtains first candidate values for one or more modifiable risk factors of the patient (604). Each modifiable risk factor defines a mutable characteristic of the patient or the arthroplasty procedure that is determined to impact the likelihood that dislocation or other complication(s) results from the arthroplasty procedure that is planned for the patient. The first candidate values are pre-operatively determined to minimize the likelihood that dislocation results from the arthroplasty procedure.

The medical user obtains second candidate values for the one or more modifiable risk factors of the patient (606). The second candidate values are determined to maximize the likelihood that dislocation or other complication(s) results from the arthroplasty procedure. Examples of the first and/or second candidate values for the modifiable risk factors can include femoral head diameter, a type of acetabular liner, a type of revised component, or a surgical approach.

The medical user enters the values for one or more non-modifiable risk factors, first candidate values, and second candidate values for the one or more modifiable risk factors of the patient into a patient-specific dislocation risk prediction model that determines dynamic risk modification based on the non-modifiable and modifiable risk factors, such as system 100. In some examples of the process 600, the system identifies a first value of the modifiable risk factors which is associated with a lowest risk, and/or a second value associated with the highest risk, of the complication from the arthroplasty procedure among all possible values for the modifiable risk factor; and selects the first value, and/or the second value, for inclusion in the first and/or second candidate values.

The system determines a personalized risk interval that represents a range of modifiable risk for the patient with respect to the arthroplasty procedure (608). This includes determining (i) a lower bound for the personalized risk interval based on the values for the one or more non-modifiable risk factors of the patient and the first candidate values for the one or more modifiable risk factors and (ii) an upper bound for the personalized risk interval based on the values for the one or more non-modifiable risk factors of the patient and the second candidate values for the one or more modifiable risk factors.

The system determining the upper and/or lower bound for the personalized risk interval can include processing, with a machine-learning model, the values for the one or more non-modifiable risk factors of the patient and the first candidate values for the one or more modifiable risk factors. Some examples of the machine-learning model include a regression model, a nomogram, an artificial neural network, a transformer model, or an XGBoost model.

The system provides an output indicative of the risk interval (610). This can include displaying an indication of the risk interval, such as to the user 102 on the interface device 104, storing the indication of the risk interval, or transmitting the indication of the risk interval to a remote computing system. The indication can be stored and/or transmitted locally, e.g., on the interface device 104, or to a remote computing system, e.g., to a networked computing system over the internet. In another example, this can include generating computer code including instructions that, when executed, cause an indication of the risk interval to be presented in an interactive user interface on a screen of an electronic device, e.g., interface device 104.

Optionally, the process 600 can include obtaining a set of user-specified values for the one or more modifiable risk factors of the patient; and determining a personalized, modifiable risk score for the patient based on the values for the one or more non-modifiable risk factors of the patient and the set of user-specified values for the one or more modifiable risk factors.

FIG. 7 is a flowchart of an example process 700 for determining a personalized, modifiable risk score for a patient, e.g., patient 106, with respect to a complication and an arthroplasty procedure based on user-indicated values for one or more non-modifiable and modifiable risk factors. In some examples, the arthroplasty procedure is a total hip arthroplasty procedure, and the complication is dislocation.

The process 700 may be used, for example, by a medical user, e.g., user 102, for determining personalized, modifiable risk score from complications for the patient undergoing the arthroplasty procedure, e.g., a total hip or other arthroplasty procedure, using a rick calculation system, e.g., system 100.

A system obtains, from the user, user-indicated values for one or more non-modifiable risk factors of a patient (702). Each non-modifiable risk factor defines an immutable patient characteristic that is determined to impact a likelihood that a complication results from the arthroplasty procedure planned for the patient.

The system obtains, from the user, user-indicated values for one or more modifiable risk factors of the patient (704). Each modifiable risk factor defines a mutable characteristic of the patient or the arthroplasty procedure that is determined to impact the likelihood that the complication results from the arthroplasty procedure. In an example, the non-modifiable risk factors include image features extracted from one or more pre-operative images of a femoral or pelvic region of the patient. Optionally, extracting the image features includes using a machine-learning model trained to predict whether, or a likelihood that, a patient exhibits dislocation following the total hip arthroplasty procedure based on one or more pre-operative images of the femoral or pelvic region.

The system determines a personalized, modifiable risk score for the patient (706). The personalized, modifiable risk score is determined with respect to the complication and the arthroplasty procedure based on the user-indicated values for the one or more non-modifiable risk factors of the patient and the user-indicated values for the one or more modifiable risk factors. Determining the personalized, modifiable risk score can include processing, with a machine-learning model (e.g., any machine-learning model described herein), the user-indicated values for the one or more non-modifiable risk factors of the patient and the user-indicated values for the one or more modifiable risk factors.

The system provides an output indicative of the personalized, modifiable risk score for the patient (708). The system can provide the output in any manner described herein.

### Example Study 1

This study involved a multimodal machine learning-based risk calculator using a single preoperative anteroposterior hip radiograph coupled with demographic, comorbidity, and surgical information predicted patient-specific dislocation risk following primary total hip arthroplasty.

### Materials and Methods

A pipeline of machine learning algorithms (image classification, feature extraction, and survival analysis) was designed to predict dislocation risk when given a patient's latest preoperative AP radiograph and clinical characteristics. Additionally, a graphical user interface, such as the system 100 of FIG. 1 operating the user interface 112 of FIGS. 2 and 3, was developed to facilitate clinical use, while ensuring model explainability.

### Study Sample Selection and Splitting

This retrospective cohort study of a total joint arthroplasty registry was Health Insurance Portability and Accountability Act compliant and approved by the institutional review board with waiver of informed consent. Patients who underwent primary THA between 1998 and 2018 were evaluated, yielding 21,978 patients whose charts were reviewed to extract demographics, relevant comorbidities, and surgical characteristics, as detailed in FIG. 8. The primary outcome was postoperative dislocation within 5 years, as dislocation becomes less common as time from surgery increases. Patients who did not have digitized preoperative AP pelvic or hip radiographs (n=4905) in our previously established THA radiograph registry were excluded, yielding a final cohort of 17,073 THA procedures. A representative test set of 10% of these patients with the same distribution of outcomes and surgery dates was randomly selected. The remainder of the dataset was separated into 10 folds at the patient level, while ensuring all folds contained approximately the same number of patients with dislocation. Tenfold cross-validation was used for hyperparameter optimization, with evaluation of the best-performing fold on the holdout test set.

### Image Classifier

Patients with definite 5-year ground truth labels (e.g., those sustaining dislocation within 5 years or who were followed up for 5 years and did not have dislocation) were used to train a multimodal binary classifier for predicting 5-year dislocation. Preoperative AP images were de-identified and their pixel values were clipped between the 2.5th and 97.5th percentile and were passed to a previously trained object detection model to detect the hip joint coordinates and side of the body shown in the image. The resulting bounding box for the joint of interest was dilated by 25% in the lateral and superior borders and was used to crop the images. Cropped images were padded to square, resized to 512×512 pixels, and standardized to have values between 0 and 1.

A hybrid network was developed that used the output of the first five blocks of an EfficientNetB4 (pretrained on ImageNet) to serve as the patch-embedding layer of a Swin-B vision transformer (randomly initialized) (FIG. 9). Both models were adapted from PyTorch image models (timm) implementation. Clinical variables were encoded (FIG. 8) and concatenated to the imaging features extracted by the transformer. This model was trained using FocalLoss as loss function and LAMB as optimizer. To account for imbalance in the training data given the rarity of dislocation as a complication, the minority class was oversampled by a factor of 15.

Although the primary interest in this study focused on patients' most recent preoperative AP pelvic and hip radiographs, using only the latest radiographs would limit the training data. To address this issue, all preoperative AP pelvic and hip radiographs were used for training, but the loss value for each image was weighted based on their temporal timestamp (TT), as follows:

$$T T = \frac{1}{{e}^{1 + \frac{d}{365}}}$$

where d is the duration between image acquisition and surgery in days. Also, the TT for each image was broadcasted and stacked as the second channel to that image (FIG. 9). Using TT as described above ensured a larger contribution of recent images to model learning (through loss weighting) and demonstrated the importance of image-to-surgery interval on the features learned (through image concatenation).

During training, images were augmented by applying rotation (615°), zooming (60.1), and horizontal flipping, using the Medical Open Network for Artificial Intelligence (e.g., MONAI) package (902). Each batch contained 128 images. Learning rate was increased from 0.0001 to 0.001 during the first 50 epochs and gradually decreased for a total of 100 epochs. Exponential moving weight averaging with a decay factor of 0.99 and weight decay of 0.0005 were used to regularize the training. Training was carried out using the PyTorch framework (v1.10.0; pytorch.org) on four NVIDIA A100 accelerators.

Although the outputs of the developed deep learning model naively could be regarded as dislocation risks, this would mean excluding from the training data the patients who had undergone less than 5 years of follow-up, leading to potential bias through changing the data distribution. Therefore, we used this model as an image feature selector and trained a final survival model on all patients' data, regardless of follow-up length.

### Image Feature Selection and Survival Analysis

The trained classifier was applied to the radiographs in all patients, including those with indefinite outcome (e.g., having undergone less than 5 years of follow-up). An output vector of the Swin transformer features (length=1024) was saved for each image (906, FIG. 9). A first extreme gradient boosting machine model (XGBoost) was then used to select the top 10 imaging features that had the most overall gain of performance on the training set across cross-validation folds (908). These 10 features were then extracted for all patients and concatenated with 21 encoded clinical features (910, encoding details in FIG. 8). This set of 31 features was used to train a second XGBoost survival model to predict the risk of dislocation for each patient, serving as the final calculator. Survival embeddings were also applied to the risk outputs of all XGBoost models to calculate 5-year hazard 912, hereafter called risk.

For model development, features were extracted from all available images for patients in the training set, but only the latest preoperative images for patients in the holdout test set were used for evaluation purposes (as is the real use case of the model). For all survival models, hyperparameters were optimized during cross-validation.

### Statistical Analysis

Categorical variables are presented as numbers with percentages in parentheses and continuous variables as medians with IQRs in parentheses or as means±SDs. To compare patient characteristics in the training and test sets, x2 test (for categorical variables) and t test (for continuous variables) were used. Area under the receiver operating characteristic curve (AUC) was used for classifier model selection and performance evaluation. Captum package (v0.4.0; ai.facebook.com) was used to visualize the 10 selected imaging features, and each neuron's global integrated gradient map was generated. The performance of the multimodal survival model was reported and compared with a naive XGBoost survival model, which was trained with only clinical data, without including imaging features (hereafter called the clinical-only model). Harrell C index, a generalization of AUC to survival data, was used to report survival model performance. The C index shows the percentage of patient pairs in which the risk of the patient with a shorter time to event is higher than that of the other patient in that pair. To gain an intuition behind survival model predictions, Shapley additive explanation (SHAP) values were calculated that show the contribution of each variable in the final output.

All metrics were reported on the latest preoperative radiograph in patients in the test set and calculated using the SciPy package (v1.7.0; https://www.python.org). CIs of the C indexes for survival models were calculated by using the survcomp package (v3.14) and compared using the compareC (v1.3.1) package in R language (R Foundation for Statistical Computing). P values less than 0.05 were considered significant.

### Results

### Study Sample Characteristics

The final study sample contained 17,073 THA procedures in 8184 men (48%) and 8889 women (52%) with a median age of 65 years (IQR: 18 years) and mean follow-up time of 4.3 years±4.0. The median number of images for each patient was four (IQR: four images). The incidence of 5-year dislocation was 2% (355 of 17,073). FIG. 10 summarizes patient characteristics in the training and test sets.

### Image Classifier

A total of 22,724 images from 129 different acquisition devices were used to train and validate the image classifier. This model achieved a mean AUC of 0.73±0.02 across the validation folds. The classifier was able to differentiate patients with implant dislocation from those who did not have dislocation within 5 years, with an AUC of 0.77 (95% CI: 0.74, 0.81) on the test set. The sensitivity of this model was 69% (25 of 36), with a specificity of 68% (469 of 685), a negative predictive value of 98% (469 of 480), and a positive predictive value of 10% (25 of 241).

Image Feature Selection and Survival Analysis The clinical-only survival model achieved a C index of 0.64 (95% CI: 0.60, 0.68) on the holdout test. In comparison, the multimodal survival model, which leveraged both clinical and imaging features, achieved a C index of 0.74 (95% CI: 0.69, 0.78; P=0.02). The SHAP summary plot of the multimodal XGBoost model and clinical-only model are presented in FIG. 11. Four of the top five and 10 of the top 13 features that influenced the multimodal survival model's predictions were imaging features. The integrated gradient maps of the 10 selected features are shown in FIG. 12, demonstrating a focus of the model on the area of maximum load in the hip joint articulation and the acetabular teardrop.

The developed graphical user interface for the multimodal calculator starts by taking all the demographic information, relevant comorbidity variables, and latest preoperative AP pelvic radiograph for a patient. It then produces a matrix of all possible outputs of patient-specific risk on the basis of the 18 combinations of modifiable surgical variables (two femoral head component sizes, three acetabular liner types, and three surgical approaches). This matrix output enables a surgeon to see the range of risk possibilities for a specific patient and the degree to which that risk can be modified by various surgical strategies. FIGS. 3A and 3B demonstrate this procedure for two patients from the test set with known outcomes, highlighting patient-specific variability of risk and how the outputs may be visualized. For example, the patient for which the table 302 was calculated in FIG. 3A has a low risk at baseline, and the model matrix demonstrates essentially no impact of surgical decisions. By contrast, the patient for which the table 310 was calculated in FIG. 3B has a higher risk, and the model matrix shows that risk can be as high as 8% versus as low as 3% based on surgical decisions. FIGS. 4 and 5 show patient-specific SHAP plots, highlighting the relative contribution of various factors to model prediction.

### Discussion

Implant dislocation is the most common complication of THA and causes serious morbidity. There are surgical choices that can reduce the risk of dislocation. A patient-specific risk prediction tool with dynamic output based on surgical decisions has remained elusive and has been a primary barrier to progress on this large-scale problem. In this study, a machine learning-based pipeline is introduced to evaluate risk of dislocation on the basis of a single preoperative radiograph in combination with clinical and surgical characteristics of patients undergoing primary THA. It showed that a fusion of imaging and clinical features was synergistic and produced optimal performance (C index, 0.74; P=0.02). The multimodal model provides individualized risk and a matrix of outputs based on surgical decisions for consideration by a surgeon preoperatively.

As dislocation is a time-to-event outcome, a single classifier would not satisfy the needs of risk prediction, therefore survival analysis methods were used in our study. Survival analysis can be considered as a type of regression task, meaning that the model, regardless of type, will output a risk or a proxy measure of it that is inversely correlated to patient time-to-event duration. More importantly, survival analysis can handle censored patients (those who had undergone less than 5 years of follow-up), but these patients should be removed for training classifiers. If two patients have dislocation in 1 year (patient A) and 3 years (patient B), a binary classifier would assign the same label to both; however, a survival model will assign a higher risk value to the patient who had dislocation in 1 year. A third patient (patient C) who was followed up for 4 years but did not have dislocation should be excluded from the classifier training pool because of the lack of a definite 5-year outcome. But a survival model should assign a lower risk to this patient, compared with patient B, as patient C definitely had no dislocation by 3 years (the time-to-event for patient B).

Our team has previously worked on a logistic regression-based risk calculator with a C index of 62% that uses demographic, past medical, and surgical variables to predict the dislocation risk of patients. That clinical calculator highlighted wide patient-specific dislocation risks (0.3%-45%), as well as the importance of modifiable surgical variables. To build upon this work, a multimodal survival model was developed that leverages both imaging and clinical data. A preoperative AP radiograph was used as the imaging input, given that it is standardized, low cost, and universally obtained prior to THA. Furthermore, this image has the potential for deriving risk scores for other THA complications, like periprosthetic femoral fracture, which could ultimately inform a more comprehensive THA risk prediction and personalized surgical planning tool.

The developed multimodal calculator outperformed the previously published logistic regression-based model by a high margin in terms of C index. It also performed better than the clinical-only gradient boosting machine survival model developed in this study, demonstrating that the observed gain of performance was not attributed to architectural differences. Also, the SHAP plots highlight the importance of imaging features in predicting dislocation risk. There were differences between the SHAP values in the clinical-only model and the final multimodal model. For example, the effect size of patient sex and spinal surgery was different in the two models. A possible explanation for this difference is that variables like sex or past surgeries might have imaging signals that are detected in a proxy fashion by the model.

The superior predictive performance of imaging features compared with most clinical features in the multimodal model may provide new insight into the root causes of dislocation risk. The integrated gradient maps of the 10 most important imaging features in the model showed the most focus on regions of anatomic interest, including the joint line at the acetabular sourcil (the most common area for joint space narrowing indicating a THA) and the acetabular teardrop. These are discrete anatomic landmarks, and they enable conjecture into how the model may be determining subtleties of risk. Perhaps the model is gaining insight into the severity of arthritis, leg length, and hip joint offset and how these parameters compare in proportion to other landmarks, though extensive efforts are needed to gain insight into how a network reaches a decision.

The convolutional layers implemented in this study were used in the same way as convolutional vision transformers and were combined with Swin transformers to boost model performance on our moderate-sized dataset. Additionally, since adding clinical data to machine learning models improves final model performance, so we used a multimodal model to better estimate patient dislocation risk.

The results of this study should be interpreted considering some limitations. First, patients from a single institution were evaluated, emphasizing the need for external testing using data from other institutions. It should be noted that our total joint registry includes data from a large population and captures every dislocation event for patients, regardless of whether they occur in our health system or not. Although images were acquired over 20 years with more than 120 different devices, extensive multi-institutional studies are required to evaluate the algorithm's generalizability. Second, as this was a retrospective study, the pipeline should be validated in prospective studies. Third, the model accounts for preoperative characteristics and imaging to determine how risk can be managed intraoperatively with surgical decisions. However, there are other elements to surgical technique that were prohibitively difficult to account for, such as implant positioning and soft tissue management. Finally, rare outcomes, especially those with multifactorial determination like THA dislocation, limit the performance of survival models. Especially, in our study, there was not a high acetabular liner diversity between patients with dislocation. This needs further exploration in future multicentric studies.

In summary, a multimodal calculator for predicting dislocation was introduced by combining preoperative characteristics and radiographic features of patients undergoing primary THA. This study highlights the superiority of imaging features compared with clinical variables and the synergy between these modes of patient evaluation. This tool enables patient-specific dislocation risk prediction with an acceptable C index, and more importantly, shows the degree to which this risk is modified by decisions within a surgeon's control. Ultimately, we envision this will underlie a powerful tool for personalized surgery to address the most common complication (dislocation) of THA surgery. Furthermore, the high performance of predicting dislocation on the basis of a single preoperative radiograph, and the resultant integrated gradient maps, provide new insights for orthopedic surgeons into the possible causes of dislocation.

### Example Study 2

### Patients and Methods

29,349 THA performed between 1998-2018 were evaluated including 21,978 primary and 7,371 revision cases. During mean 6-year follow-up, 1,521 THA sustained a dislocation. Patients were characterized through individual chart review on non-modifiable factors (e.g., demographics, THA indication, spinal disease, spine surgery, neurologic disease), and modifiable operative decisions (e.g., operative approach, femoral head diameter, acetabular liner [standard/elevated/constrained/dual mobility]). Multivariable regression models and nomograms were developed with dislocation as a binary outcome at 1-year and 5-years postoperatively.

### Patients

Following Institutional Review Board approval, 29,349 THA performed at a single institution from 1998-2018 were evaluated including 21,978 primary and 7,371 revision cases with a mean 6 years of follow-up. Patients were characterized using a prospectively-collected total joint registry with augmentation to determine specific “instability comorbidities” of interest (e.g., neurologic disease, spinal disease, spine surgery) using diagnosis/procedure codes and natural language processing (NLP)-assisted chart review of the medical record with individual manual review of all diagnoses. Ultimately, this enabled determination of patient profiles with non-modifiable preoperative variables and modifiable intraoperative variables. Included variables are detailed in FIG. 13. All cases were assumed to be at risk of dislocation after THA and were followed until dislocation, last follow-up, or death. Cox regression analysis determined hazard ratios (HRs) for variables associated with differential dislocation risk.

A patient-specific dislocation risk calculator was created with nomograms from multivariable modeling such that the individual risk for a patient with any combination of non-modifiable factors could be calculated and would determine differential risk based on modifiable operative decisions. These nomograms were built separately for primary and revision cohorts for both 1-year and 5-year timepoints. Discrimination was assessed using the concordance statistic (c-statistic) for the Cox models. Calibration was assessed by comparing observed versus expected events in deciles of predicted risk using goodness-of-fit tests, which included standardized incidence ratios (SIR). All hazard ratios reported in Results are statistically significant, with confidence intervals and p-values reported in the figures.

### Patient Characteristics and Operative Management

Baseline characteristics are summarized in FIG. 14 for the overall cohort as well as the primary and revision subgroups. Overall, mean patient age was 65 years (range, 18-100 years), mean BMI was 30 kg/m² (range, 13-82 kg/m²), and 52% patients were female. Mean follow-up was 5.5 years (range, 2-21 years). History of minor spine disease, major spine disease, minor spine surgery and major spine surgery were present in 37%, 7%, 4% and 1% of patients, respectively. Neurologic disease was present in 19% of patients (FIG. 14).

In primary THA, surgery was performed for osteoarthritis (88%), osteonecrosis (6%), post-traumatic (2%), inflammatory (1%), and other (2%; top 3 diagnoses included dysplasia, skeletal dyscrasias, neoplasm). In revision THA, surgery was performed for aseptic loosening (50%), periprosthetic joint infection (15%), wear/osteolysis (13%), dislocation (13%), and periprosthetic fracture/nonunion (10%). The acetabular component was revised in 60% of cases (FIG. 14).

In primary THA, 50% were performed with posterior approach, 35% with lateral approach, 13% with direct anterior approach, and 1% with trochanteric osteotomies. Femoral heads ≥36 mm were utilized in 50% and ≤32 mm in 50%. Standard flat liners were used in 92%, elevated/face changing liners in 7%, and dual mobility in 2%. In revision THA, 47% were performed with a lateral approach, 37% with a posterior approach, 16% with trochanteric osteotomies, and 0.4% with a direct anterior approach. Femoral heads ≤32 mm were utilized in 60% and ≥36 mm in 40%. Standard flat liners were used in 70%, elevated/face changing liners in 13%, constrained in 11%, and dual mobility in 7% (FIG. 14).

### Results

Among the 29,349 THAs, 1,521 sustained a postoperative dislocation (2.9% primary, 12.1% revision, 5.2% overall). Univariable and multivariable analyses are summarized in Table 3. In primary THA, the following factors were significantly associated with increased dislocation risk: trochanteric osteotomies (HR≥1.78), “other” as an indication for THA versus osteoarthritis (HR=1.62), major spine disease (HR=1.52), and neurologic disease (HR=1.52). The following factors were associated with decreased dislocation risk: direct anterior approach and lateral approach versus posterior approach (HR=0.27, HR=0.58), elevated liners (HR=0.63), ≥36 mm femoral heads (HR=0.69), male sex (HR=0.73), and increasing age in 10-year increments (HR=0.92) (FIG. 15).

In revision THA, the following factors were associated with increased dislocation risk: instability of previous THA, infection, or periprosthetic fracture as the indications for revision compared to aseptic loosening (HR=1.89, HR=1.69, HR=1.38), major spine disease (HR=1.39), BMI 35.0-39.9 kg/m² (HR=1.37), neurologic disease (HR=1.33), and osteonecrosis as the underlying diagnosis (HR=1.31). The following factors were associated with decreased dislocation risk: dual mobility constructs (HR=0.44), trochanteric osteotomies (HR=0.56), constrained acetabular liners (HR=0.67), elevated liners (HR=0.74), male sex (HR=0.80), and ≥36 mm femoral heads (HR=0.82) (FIG. 15). Furthermore, acetabular component revision decreased risk of dislocation similarly for both isolated acetabular revision (HR=0.58) and in combination with femoral revision (HR=0.59) (FIG. 16). Acetabular revision remained protective across all indications for revision (FIG. 16).

The multivariable models demonstrated calibration SIR values of 0.96-1.02, consistent with “excellent” calibration. C-statistic discrimination ranged from 0.63-0.65, consistent with “good” discrimination.

### Nomogram Risk Calculator and Spectrum of Individual Patient Risk

Nomograms of individual patient dislocation risk were created from Cox proportional hazard models (FIGS. 17A-17D). Each patient factor is calibrated to be worth a certain number of points. Total points are calculated to obtain projected risk of dislocation at 1-year and 5-years for primary or revision. Revision THA nomograms include acetabular revision as a modifiable operative factor. The final data input line in the nomograms is approach, head diameter, and liner. The combination of these 3 factors yields the greatest differential in possible point total, underscoring the power surgeons have to modify risk.

To understand the range of risk associated with non-modifiable patient factors, as well as the impact of modifiable operative decisions, a series of patient scenarios were created to define the upper and lower boundaries of the nomogram using best and worst possible patient scenarios (FIG. 18). The nomograms demonstrated that patient-specific dislocation risk was wide-ranging, from 0.3%-13% at 1-year and 0.4%-19% at 5-years in primary THA, and 2%-32% at 1-year and 3%-42% at 5-years in revision THA. In primary THA, when adjusting for approach, the combination of femoral heads ≥36 mm and elevated liners yielded the largest decrease in risk (HR=0.28), followed by dual mobility constructs (HR=0.48). In revision THA, the adjusted risk of dislocation was most markedly decreased with dual mobility constructs (HR=0.40), followed by femoral heads ≥36 mm and elevated liners (HR=0.88).

### Case Examples

Sixty-nine year old female, BMI 36 kg/m², THA for osteoarthritis, and a positive history of neurologic disease and major spinal disease/surgery. Her absolute risk of dislocation at 1-year ranges from 1.2%-8.4%, based on choices within control of the surgeon. Using the highest risk as a reference, but maintaining posterior approach, the 1-year risk decreases from 8.4% to 6.1% with use of a ≥36 mm head and decreases to 2.5% with use of a ≥36 mm head and elevated liner. By contrast, use of dual mobility in this patient reduces risk to 4.1% (FIG. 19A). Furthermore, risk for this patient using a lateral or direct anterior approach can be followed in FIG. 18 or FIG. 19A.

Presume the same patient presents for revision THA due to instability. Her absolute risk of dislocation at 1-year ranges from 11.8%-28%. Using the highest risk as a reference, but maintaining posterior approach, the 1-year risk decreases from 28% to 24.6% with use of a ≥36 mm head and decreases to 25.4% with use of a ≥36 mm head and elevated liner. By contrast, use of a dual mobility construct in this situation decreases risk to 11.8% (FIG. 19B). If there is an indication to revise the acetabular component for this patient (or the surgeon chooses to for stability optimization) the relative risk of dislocation for any combination of approach/liner/head diameter is further decreased by 40%.

### Discussion

Dislocation remains one of the most frequent complications and reasons for revision following THA. Ultimately, individual patient risk is a complex amalgamation of non modifiable characteristics and modifiable operative decisions. This study leveraged a large cohort of patients that was meticulously characterized across a broad range of important dislocation risk comorbidities to derive risk prediction nomograms that are patient-specific and responsive to operative decisions. Surgeons can use these prediction tools to forecast 1-year and 5-year probability of dislocation and determine the impact of implant and operative approach for dislocation risk mitigation.

It should be emphasized that this cohort included thorough characterization of patients beyond traditional demographic and operative factors by including other “instability comorbidities” including spine disease, spine surgery, and neurologic disease. We further characterized spine disease and spine surgery separately for those inducing either a biological or operative fusion to account for the stiff spine that has become increasingly recognized as a factor in instability. This data was all individually and manually validated. All included factors were highly influential in the multivariable model. The aforementioned diagnoses then supplemented our total joint registry that already tracks patient demographic, operative, and complication data with >98% capture.

Baseline risk of dislocation was shown to be highly variable based on non-modifiable comorbidities and risk factors, with all factors included in the final model impacting the risk of dislocation, a fact most poignantly demonstrated by comparing “worst case” and “best case” in FIG. 18. This underscores the complex nature of THA stability and highlights the importance of comprehensively assessing comorbid status to accurately classify patients. Nevertheless, an encouraging message for surgeons in the presented work centers on the considerable control to influence outcomes based on operative decisions. Indeed, operative covariates were the most impactful nomogram variables by a wide margin. Operative approach demonstrated a marked influence on dislocation risk, consistent with previous literature. For surgeons that perform some combination of approaches in practice, this may afford an opportunity to more selectively employ one approach versus another. However, for the many surgeons who default to a specific approach, the data provides highly actionable information as well. For example, a predominantly posterior approach surgeon can see in FIG. 19A that changing from a 32 mm to a 36 mm femoral head decreases risk of dislocation by 30%, and adding an elevated liner further decreases risk by 70%. Similarly, if it is not possible to upsize to a 36 mm femoral head, an isolated change to an elevated liner decreases risk by 45%. The relationship between head size and dislocation protection was similar across all 3 operative approaches. We also noted in the revision THA population that acetabular revision markedly decreased the risk of dislocation (40%) if performed in isolation or concomitantly with femoral revision. This relationship was consistent across all indications for revision. Revising the acetabular component yields intuitive advantages for mitigating dislocation risk. First, it affords an opportunity to improve component position. Second, it usually leads to a larger acetabular component which may enable larger diameter heads or more diverse liner options. We also noted that performance of a trochanteric osteotomy in revision THA decreased risk by 43% (FIG. 15). This was not included in the nomograms as proximal femoral osteotomies are typically undertaken out of necessity, thus would not be considered a modifiable operative decision. Nevertheless, the protective effect is interesting to note and may relate to maintenance of proximal soft tissues, ability to tension the abductors, and protected weight bearing and bracing posteroperatively.

It should be noted that absolute risk is important to consider in addition to relative risk. The overall impact of a mitigation strategy is quite different for a patient with a baseline 1-year dislocation risk of 2% versus 16%. In this example, undertaking an operative decision that reduces relative risk by 50% changes the absolute risk from 2% to 1% vs 16% to 8% in these hypothetical patients. This fact emphasizes the importance of determining patient-specific baseline risk, which this study shows is highly variable and subject to many demographic and comorbid influences.

Another increasingly common question among surgeons is when to use a dual mobility construct. Several studies have demonstrated decreased risk of dislocation with these implants. However, dual mobility comes with increased cost and potential unique complications such as intraprosthetic dislocation, malseating, and corrosion. Therefore, until more data becomes available, judicious use is likely warranted. In aggregate, dual mobility constructs showed a roughly 50% decrease in risk of dislocation in the primary THA cohort, but importantly, this was not superior to the use of larger heads and elevated liners. However, use of dual mobility constructs in the primary setting was relatively rare in this cohort (N=354; 1.6%) and trends toward increased use may alter this data in future studies. Furthermore, there was wide discrepancy for risk modification in univariable analysis (HR=1.08) versus multivariable analysis (HR=0.59), suggesting these implants were used in complex patients. By contrast, dual mobility constructs were the most influential risk mitigator in revision THA with HR=0.40 (FIG. 20). Therefore, the data is more supportive of dual mobility use in revision cases with benefits attenuated in the primary setting compared to other implant-related risk mitigation strategies.

Some may be surprised by the high incidence of dislocation in this study. However, these rates are consistent with previous reports from our institution that not only tracks patients clinically at routine intervals, but also incorporates phone and mail communications to document any complications that have been managed at other facilities. Thus, these rates of dislocation, although higher than many reports, probably are more representative of the true complication burden, which further underscores the importance of risk mitigation. Indeed, a recent study from the Danish Arthroplasty Registry documented a 3.5% “true” incidence of dislocation within 2 years of THA, which is even higher than we report. The authors conclude that most studies to date have systematically underreported dislocation incidence as most dislocations are treated in emergency departments without surgery.

This study can be interpreted in light of potential limitations. First, although the nomograms were derived using robust, manually-validated clinical data on patient characteristics, they are not comprehensive. Notably, implant position is not considered given constraints in a cohort this large. Secondly, dislocation is a relatively rare event with multifactorial etiology. That combination, for any clinic prediction problem, makes model discrimination and calibration difficult. Our model had “good” discrimination, which is not unexpected given the aforementioned challenges inherent to modelling problems like dislocation. However, we did achieve “excellent” calibration, which is a testament to model fine-tuning. Thirdly, these results represent a single center experience and have not been externally validated. This will be a critical step to broader acceptance and generalizability. Fourth, “elevated liners” included face-changing/lateralied/lipped. Sample size precluded evaluating these liners individually.

This study is the first to our knowledge yielding a patient-specific dislocation risk calculator that incorporates important comorbidities for instability. The resultant nomograms are responsive to implant and operative approach decisions, and thus can be used as a screening tool to identify and individualize recommendations and treatment for THA patients. This is especially important given the wide range of individual patient risk identified in this study, and the degree of risk mitigation portended by various operative strategies. Nomograms from this work will serve as the underlying foundation for a digital clinical tool to calculate patient risk in a streamlined fashion.

FIG. 21 shows an example computer system 2100 on which the patient-specific dislocation risk prediction system 100 can be hosted that includes a processor 2100, a memory 2120, a storage device 2130 and an input/output device 2140. Each of the components 2110, 2120, 2130 and 2140 can be interconnected, for example, by a system bus 2150. The processor 2110 is capable of processing instructions for execution within the system 2100. In some implementations, the processor 2110 is a single-threaded processor, a multi-threaded processor, or another type of processor. The processor 2110 is capable of processing instructions stored in the memory 2120 or on the storage device 2130. The memory 2120 and the storage device 2130 can store information within the system 2100.

The input/output device 2140 provides input/output operations for the system 2100. In some implementations, the input/output device 2140 can include one or more of a network interface device, e.g., an Ethernet card, a serial communication device, e.g., an RS-232 port, and/or a wireless interface device, e.g., an 802.11 card, a 3G wireless modem, a 4G wireless modem, a 14G wireless modem, etc. In some implementations, the input/output device can include driver devices configured to receive input data and send output data to other input/output devices, e.g., keyboard, printer and display devices 2160. In some implementations, mobile computing devices, mobile communication devices, and other devices can be used.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device. While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1-24. (canceled)

25. A computer-implemented method, comprising:
obtaining values for one or more non-modifiable risk factors of a patient, each non-modifiable risk factor defining an immutable patient characteristic that is determined to impact a likelihood that a dislocation results from an arthroplasty procedure that is planned for the patient;
obtaining first candidate values for one or more modifiable risk factors of the patient, each modifiable risk factor defining a mutable characteristic of the patient or the arthroplasty procedure that is determined to impact the likelihood that the dislocation results from the arthroplasty procedure that is planned for the patient, wherein the first candidate values are determined to minimize the likelihood that the dislocation results from the arthroplasty procedure;
obtaining second candidate values for the one or more modifiable risk factors of the patient, wherein the second candidate values are determined to maximize the likelihood that the dislocation results from the arthroplasty procedure;
determining a personalized risk interval that represents a range of modifiable risk for the patient with respect to the arthroplasty procedure, including determining (i) a lower bound for the personalized risk interval based on the values for the one or more non-modifiable risk factors of the patient and the first candidate values for the one or more modifiable risk factors and (ii) an upper bound for the personalized risk interval based on the values for the one or more non-modifiable risk factors of the patient and the second candidate values for the one or more modifiable risk factors; and
providing an output indicative of the personalized risk interval.

26. The computer-implemented method of claim 25, wherein:
determining the lower bound for the personalized risk interval comprises processing, with a machine-learning model, the values for the one or more non-modifiable risk factors of the patient and the first candidate values for the one or more modifiable risk factors; and
determining the upper bound for the personalized risk interval comprises processing, with the machine-learning model, the values for the one or more non-modifiable risk factors of the patient and the second candidate values for the one or more modifiable risk factors.

27. The computer-implemented method of claim 26, wherein the machine-learning model comprises at least one of a regression model, an artificial neural network, a transformer model, or an XGBoost model.

28. The computer-implemented method of claim 25, wherein the arthroplasty procedure is a total hip arthroplasty procedure and the one or more non-modifiable risk factors comprise at least one of a THA indication of the patient, a sex of the patient, a body mass index of the patient, an age of the patient, an indication of a neurologic disease of the patient, a diagnosis of spine disease of the patient, a history for spine surgery of the patient, a surgery indication of the patient, or a comorbidity of the patient.

29. The computer-implemented method of claim 28, wherein:
the sex of the patient is associated with values that include male and female;
the age of the patient is associated with values that include a number of years or decades since the patient's birth;
the THA indication of the patient is associated with values that include primary THA or revision THA;
the body mass index of the patient is associated with values that include less than or equal to 18, greater than 18 and less than or equal to 25, greater than 25 and less than or equal to 30, greater than 30 and less than or equal to 35, greater than 35 and less than or equal to 40, or greater than 40;
the indication of a neurologic disease is associated with values that include Parkinson disease, dementia, alcoholism, or fibromyalgia; and
the surgery indication of the patient is associated with values that include osteoarthritis, osteonecrosis, inflammation, posttraumatic, or nonunion.

30. The computer-implemented method of claim 25, wherein the arthroplasty procedure is a total hip arthroplasty procedure and the one or more modifiable risk factors comprise at least one of a femoral head diameter, a type of acetabular liner, a type of revised component, or a surgical approach.

31. The computer-implemented method of claim 30, wherein:
the femoral head diameter is associated with values of cemented and non-cemented;
the surgical approach is associated with values of posterior, lateral, direct anterior, or trochanteric osteotomy;
the type of acetabular liner is associated with values of standard, elevated, constrained, or dual-mobility; and
the type of revised component is associated with values of acetabular component, femoral component, or both components.

32. The computer-implemented method of claim 25, comprising for each of the one or more modifiable risk factors:
identifying a first value of the modifiable risk factor associated with a lowest risk of the dislocation from the arthroplasty procedure among all possible values for the modifiable risk factor; and
selecting the first value for inclusion in the first candidate values.

33. The computer-implemented method of claim 32, comprising for each of the one or more modifiable risk factors:
identifying a second value of the modifiable risk factor associated with a highest risk of the dislocation from the arthroplasty procedure among all possible values for the modifiable risk factor; and
selecting the second value for inclusion in the second candidate values.

34. The computer-implemented method of claim 25, wherein the patient is a human.

35. The computer-implemented method of claim 25, comprising:
obtaining a set of user-specified values for the one or more modifiable risk factors of the patient; and
determining a personalized, modifiable risk score for the patient based on the values for the one or more non-modifiable risk factors of the patient and the set of user-specified values for the one or more modifiable risk factors.

36. The computer-implemented method of claim 25, wherein providing the output indicative of the personalized risk interval comprises displaying an indication of the personalized risk interval, storing the indication of the personalized risk interval, or transmitting the indication of the personalized risk interval to a remote computing system.

37. The computer-implemented method of claim 25, wherein providing the output indicative of the personalized risk interval comprises generating computer code comprising instructions that, when executed, cause an indication of the personalized interval to be presented in an interactive user interface on a screen of an electronic device.

38. The computer-implemented method of claim 25, wherein the arthroplasty procedure is a total hip arthroplasty procedure and the one or more non-modifiable risk factors comprise image features extracted from one or more pre-operative images of a femoral or pelvic region of the patient.

39. The computer-implemented method of claim 38, comprising extracting the image features using a machine-learning model trained to predict whether or a likelihood that a patient exhibits dislocation following the total hip arthroplasty procedure based on one or more pre-operative images of the femoral or pelvic region.

40. A computer-implemented method, comprising:
obtaining user-indicated values for one or more non-modifiable risk factors of a patient, each non-modifiable risk factor defining an immutable patient characteristic that is determined to impact a likelihood that a dislocation results from an arthroplasty procedure that is planned for the patient;
obtaining user-indicated values for one or more modifiable risk factors of the patient, each modifiable risk factor defining a mutable characteristic of the patient or the arthroplasty procedure that is determined to impact the likelihood that the dislocation results from the arthroplasty procedure that is planned for the patient;
determining a personalized, modifiable risk score for the patient with respect to the dislocation and the arthroplasty procedure based on the user-indicated values for the one or more non-modifiable risk factors of the patient and the user-indicated values for the one or more modifiable risk factors; and
providing an output indicative of the personalized, modifiable risk score for the patient.

41. The computer-implemented method of claim 40, wherein:
determining the personalized, modifiable risk score comprises processing, with a machine-learning model, the user-indicated values for the one or more non-modifiable risk factors of the patient and the user-indicated values for the one or more modifiable risk factors.

42. The computer-implemented method of claim 40, wherein the arthroplasty procedure is a total hip arthroplasty procedure and the one or more non-modifiable risk factors comprise at least one of a THA indication of the patient, a sex of the patient, a body mass index of the patient, an age of the patient, an indication of a neurologic disease of the patient, a diagnosis of spine disease of the patient, a history for spine surgery of the patient, a surgery indication of the patient, or a comorbidity of the patient.

43. The computer-implemented method of claim 40, wherein the arthroplasty procedure is a total hip arthroplasty procedure and the one or more non-modifiable risk factors comprise image features extracted from one or more pre-operative images of a femoral or pelvic region of the patient.

44. The computer-implemented method of claim 43, comprising extracting the image features using a machine-learning model trained to predict whether or a likelihood that a patient exhibits dislocation following the total hip arthroplasty procedure based on one or more pre-operative images of the femoral or pelvic region.
